(19) United States
(12) Patent Application Publication
(10) Pub. No.: US 2024/0011937 A1
(43) Pub. Date: Jan. 11, 2024

(54) SENSOR ELEMENT AND GAS SENSOR

(71) Applicant: NGK INSULATORS, LTD., Nagoya (JP)

(72) Inventors: Takayuki SEKIYA, Nisshin (JP); Yusuke WATANABE, Nagoya (JP); Kodai ICHIKAWA, Nagoya (JP)

(21) Appl. No.: 18/472,514

(22) Filed: Sep. 22, 2023

Related U.S. Application Data
(63) Continuation of application No. PCT/JP2022/014338, filed on Mar. 25, 2022.

(30) Foreign Application Priority Data
Mar. 31, 2021 (JP) 2021-059120

(51) Int. Cl. G01N 27/409; G01N 33/00; G01N 27/41

(52) U.S. Cl. CPC G01N 27/409; G01N 33/0027; G01N 27/41

(57) ABSTRACT

A sensor element is for detecting a specific gas concentration in a measurement-object gas, and includes: an element body including an oxygen-ion-conductive solid electrolyte layer and internally provided with a measurement-object gas flow portion that introduces the measurement-object gas and causes the measurement-object gas to flow therethrough; a flow portion pump cell having a pump inner electrode disposed in an internal cavity of the measurement-object gas flow portion, the flow portion pump cell being configured to pump out oxygen from the internal cavity or pump oxygen into the internal cavity; and a flow portion sensor cell having a voltage inner electrode disposed in the internal cavity, the flow portion sensor cell being configured to generate a voltage based on an oxygen concentration in the internal cavity.

### CROSS-REFERENCE TO RELATED APPLICATION

This application is a continuation application of PCT/JP2022/014338, filed on Mar. 25, 2022, which claims the benefit of priority of Japanese Patent Application No. 2021-059120, filed on Mar. 31, 2021, the entire contents of which are incorporated herein by reference.

### BACKGROUND OF THE INVENTION

### 1. Field of the Invention

The present invention relates to a sensor element and a gas sensor.

### 2. Description of the Related Art

Hitherto, a known gas sensor detects the concentration of a specific gas, such as NOx, in a measurement-object gas, such as the exhaust gas of an automobile. For example, Patent Literature 1 describes a gas sensor including an elongate plate-shaped sensor element obtained by stacking a plurality of oxygen-ion-conductive solid electrolyte layers.

A schematic cross-sectional view schematically illustrating an example of the configuration of a gas sensor 900 in such a related art is illustrated in FIG. 17. As illustrated, the gas sensor 900 includes a sensor element 901. The sensor element 901 is an element having a structure in which oxygen-ion-conductive solid electrolyte layers 911 to 916 are stacked. In the sensor element 901, a measurement-object gas flow portion that introduces a measurement-object gas is formed between the lower surface of the solid electrolyte layer 916 and the upper surface of the solid electrolyte layer 914, and the measurement-object gas flow portion is provided with a first internal cavity 920, a second internal cavity 940, and a third internal cavity 961. An inner pump electrode 922 is disposed in the first internal cavity 920, an auxiliary pump electrode 951 is disposed in the second internal cavity 940, and a measurement electrode 944 is disposed in the third internal cavity 961. In addition, an outer pump electrode 923 is disposed on the upper surface of the solid electrolyte layer 916. In contrast, between the upper surface of the solid electrolyte layer 913 and the lower surface of the solid electrolyte layer 914, a reference electrode 942 is disposed which is in contact with a reference gas (e.g., atmospheric gas) serving as a reference for detecting a specific gas concentration in a measurement-object gas. A main pump cell 921 is formed by the inner pump electrode 922, the outer pump electrode 923, and the solid electrolyte layers 914 to 916. A measurement pump cell 941 is formed by the measurement electrode 944, the outer pump electrode 923, and the solid electrolyte layer 914 to 916. A measurement-pump-control oxygen-partial-pressure detection sensor cell 982 is formed by the measurement electrode 944, the reference electrode 942, and the solid electrolyte layers 914, 913. A Vref detection sensor cell 983 is formed by the outer pump electrode 923, the reference electrode 942, and the solid electrolyte layers 913 to 916. A reference-gas adjustment pump cell 990 is formed by the outer pump electrode 923, the reference electrode 942, and the solid electrolyte layers 913 to 916. In the gas sensor 900, when a measurement-object gas is introduced into the measurement-object gas flow portion, oxygen is pumped out or pumped in between the first internal cavity 920 and the outside of the sensor element by the main pump cell 921, and oxygen is further pumped out or pumped in between the second internal cavity 940 and the outside of the sensor element to adjust the oxygen concentration in the measurement-object gas flow portion. NOx in the measurement-object gas after adjustment of the oxygen concentration is reduced in the periphery of the measurement electrode 944. A voltage Vp2 applied to the measurement pump cell 941 is feedback-controlled so that voltage V2 generated in the measurement-pump-control oxygen-partial-pressure detection sensor cell 982 reaches a predetermined target value, thus the measurement pump cell 941 pumps out the oxygen in the periphery of the measurement electrode 944. The NOx concentration in the measurement-object gas is detected based on the pump current Ip2 which flows through the measurement pump cell 941 then. The reference-gas adjustment pump cell 990 pumps oxygen into the periphery of the reference electrode 942 by passing a pump current Ip3 by a voltage Vp3 applied across the reference electrode 942 and the outer pump electrode 923. Thus, when the oxygen concentration of the reference gas in the periphery of the reference electrode 942 decreases, the decrease in the oxygen concentration can be compensated, and reduction in the accuracy of detection of the specific gas concentration is prevented. Furthermore, a voltage Vref is generated between the outer pump electrode 923 and the reference electrode 942 in the Vref detection sensor cell 983. The voltage Vref makes it possible to detect the oxygen concentration in the measurement-object gas outside the sensor element 901.

### CITATION LIST

### Patent Literature

PTL 1: WO 2020/004356 A1

### SUMMARY OF THE INVENTION

Meanwhile, further improvement of the accuracy of detection of an oxygen concentration has been demanded for detection of the oxygen concentration in the internal cavity of the measurement-object gas flow portion using the voltage of a sensor cell, such as the voltage V2 of the aforementioned measurement-pump-control oxygen-partial-pressure detection sensor cell 982.

The present invention has been made to solve the aforementioned problem, and a main object thereof is to improve the accuracy of detection of the oxygen concentration in the internal cavity of the sensor element using a flow portion sensor cell.

In order to achieve the aforementioned main object, the present invention employs the following solutions.

A sensor element of the present invention is for detecting a specific gas concentration in a measurement-object gas, and includes: an element body including an oxygen-ion-conductive solid electrolyte layer and internally provided with a measurement-object gas flow portion that introduces a measurement-object gas and causes the measurement-object gas to flow therethrough; a flow portion pump cell having a pump inner electrode disposed in an internal cavity of the measurement-object gas flow portion, the flow portion pump cell being configured to pump out oxygen from the internal cavity or pump oxygen into the internal cavity; and a flow portion sensor cell having a voltage inner electrode disposed in the internal cavity, the flow portion sensor cell being configured to generate a voltage based on an oxygen concentration in the internal cavity.

The sensor element includes: a flow portion pump cell to pump out oxygen from the internal cavity or pump oxygen into the internal cavity; and a flow portion sensor cell that generates a voltage based on the oxygen concentration in the internal cavity. In the internal cavity, a pump inner electrode constituting part of the flow portion pump cell; and a voltage inner electrode constituting part of the flow portion sensor cell are both disposed. In other words, in the sensor element, the pump inner electrode and the voltage inner electrode are separately provided in the one internal cavity. Thus, unlike when one electrode serves as the pump inner electrode as well as the voltage inner electrode (e.g., in the sensor element 901 illustrated in FIG. 17, the measurement electrode 944 serves as the electrode of the measurement pump cell 941 as well as the electrode of the measurement-pump-control oxygen-partial-pressure detection sensor cell 982), a pump current at the time of pumping-out or pumping-in of oxygen performed by the flow portion pump cell does not flow through the voltage inner electrode. Therefore, the voltage of the flow portion sensor cell does not include a voltage drop of the voltage inner electrode due to a pump current. Thus, the voltage of the flow portion sensor cell has a value which corresponds with higher accuracy to the oxygen concentration in the internal cavity, thus the accuracy of detection of the oxygen concentration in the internal cavity using a flow portion sensor cell is improved.

In this situation, the flow portion pump cell serves as a pumping-out destination of oxygen from the internal cavity or a pumping-in source of oxygen into the internal cavity, and may have a pump electrode provided other than the measurement-object gas flow portion. The pump electrode may be a pump outer electrode provided outside the element body so as to be in contact with the measurement-object gas. Alternatively, the flow portion sensor cell may have a reference electrode disposed inside the element body so as to be in contact with a reference gas serving as a reference for detecting the specific gas concentration.

The sensor element of the present invention may further include an adjustment chamber pump cell that adjusts an oxygen concentration in an oxygen concentration adjustment chamber of the measurement-object gas flow portion. The internal cavity may be a measurement chamber provided downstream of the oxygen concentration adjustment chamber in the measurement-object gas flow portion, the pump inner electrode may be a pump measurement electrode disposed in the measurement chamber, the voltage inner electrode may be a voltage measurement electrode disposed in the measurement chamber, the flow portion pump cell may be a measurement pump cell that pumps out oxygen produced from the specific gas in the measurement chamber, and the flow portion sensor cell may be a measurement sensor cell that generates a voltage based on an oxygen concentration in the measurement chamber. In this manner, the pump measurement electrode and the voltage measurement electrode are separately provided in one measurement chamber, thus the voltage of the measurement sensor cell has a value which corresponds with higher accuracy to the oxygen concentration in the measurement chamber, and consequently, the accuracy of detection of the oxygen concentration in the measurement chamber using a measurement sensor cell is improved. For example, use of the voltage of the measurement sensor cell to control the measurement pump cell effects on the accuracy of detection of the specific gas concentration in the measurement-object gas. Thus, the accuracy of detection of the specific gas concentration is improved by improving the accuracy of detection of the oxygen concentration in the measurement chamber using a measurement sensor cell.

The sensor element of the present invention may further include a measurement pump cell that pumps out oxygen from the measurement chamber of the measurement-object gas flow portion, the oxygen being produced from the specific gas in the measurement chamber. The internal cavity may be an oxygen concentration adjustment chamber provided upstream of the measurement chamber in the measurement-object gas flow portion, the pump inner electrode may be pump adjustment electrode disposed in the oxygen concentration adjustment chamber, the voltage inner electrode may be a voltage adjustment electrode disposed in the oxygen concentration adjustment chamber, the flow portion pump cell may be an adjustment chamber pump cell that adjusts an oxygen concentration in the oxygen concentration adjustment chamber, and the flow portion sensor cell may be an adjustment chamber sensor cell that generates a voltage based on the oxygen concentration in the oxygen concentration adjustment chamber. In this manner, the voltage of the adjustment chamber sensor cell has a value which corresponds with higher accuracy to the oxygen concentration in the oxygen concentration adjustment chamber, thus the accuracy of detection of the oxygen concentration in the oxygen concentration adjustment chamber using the adjustment chamber sensor cell is improved.

In the sensor element including the pump adjustment electrode and the voltage adjustment electrode according to an aspect of the present invention, the oxygen concentration adjustment chamber may have the first internal cavity provided in the measurement-object gas flow portion, and the second internal cavity provided downstream of the first internal cavity in the measurement-object gas flow portion, the pump adjustment electrode may be a pump main electrode disposed in the first internal cavity, the voltage adjustment electrode may be a voltage main electrode disposed in the first internal cavity, the adjustment chamber pump cell may be a main pump cell that adjusts the oxygen concentration in the first internal cavity, and the adjustment chamber sensor cell may be a first internal cavity sensor cell that generates a voltage based on the oxygen concentration in the first internal cavity.

In the sensor element including the pump adjustment electrode and the voltage adjustment electrode according to an aspect of the present invention, the oxygen concentration adjustment chamber may have the first internal cavity provided in the measurement-object gas flow portion, and the second internal cavity provided downstream of the first internal cavity in the measurement-object gas flow portion, the pump adjustment electrode may be a pump auxiliary electrode disposed in the second internal cavity, the voltage adjustment electrode may be a voltage auxiliary electrode disposed in the second internal cavity, the adjustment chamber pump cell may be an auxiliary pump cell that adjusts the oxygen concentration in the second internal cavity, and the adjustment chamber sensor cell may be a second internal cavity sensor cell that generates a voltage based on the oxygen concentration in the second internal cavity.

The sensor element of the present invention may further include: a reference-gas introduction portion disposed inside the element body, a reference-gas introduction portion being configured to introduce a reference gas serving as a reference for detecting a specific gas concentration in the measurement-object gas; and a reference-gas adjustment pump cell having a pump reference electrode disposed inside the element body so as to be in contact with the reference gas introduced to the reference-gas introduction portion, the reference-gas adjustment pump cell being configured to pump oxygen into a periphery of the pump reference electrode. The flow portion sensor cell may have a voltage reference electrode disposed inside the element body so as to be in contact with the reference gas introduced to the reference-gas introduction portion. In this manner, the reference-gas adjustment pump cell pumps oxygen into the periphery of the pump reference electrode, thus reduction in the oxygen concentration of the reference gas in the reference-gas introduction portion can be supplemented. In the flow portion sensor cell, a voltage based on the oxygen concentration difference between the reference gas and the internal cavity is generated, thus the oxygen concentration in the periphery of the voltage inner electrode can be detected with the voltage of the flow portion sensor cell. In the sensor element, the pump reference electrode and the voltage reference electrode are separately provided as electrodes to be in contact with the reference gas in the reference-gas introduction portion. Thus, unlike when one electrode serves as the pump reference electrode as well as the voltage reference electrode, a pump current at the time of pumping-in of oxygen performed by the reference-gas adjustment pump cell does not flow through the voltage reference electrode, thus the voltage of the flow portion sensor cell does not include a voltage drop of the voltage reference electrode due to a pump current. Consequently, in the sensor element, it is possible to prevent reduction in the accuracy of detection of the oxygen concentration in the internal cavity due to a pump current at the time of pumping-in of oxygen, while oxygen is being pumped into the reference-gas introduction portion. As described above, the voltage of the flow portion sensor cell does not include a voltage drop of the voltage inner electrode. Specifically, the voltage of the flow portion sensor cell is the voltage across the voltage inner electrode and the voltage reference electrode, and no pump current flows through each of the voltage inner electrode and the voltage reference electrode. Therefore, the voltage of the flow portion sensor cell has a value which corresponds with higher accuracy to the oxygen concentration in the internal cavity.

In this situation, the reference-gas adjustment pump cell serves as a pumping-in source of oxygen to the periphery of the pump reference electrode, and may have a pumping-in source electrode disposed inside or outside the element body so as to be in contact with the measurement-object gas. In addition, the reference-gas adjustment pump cell may pump out oxygen from the periphery of the pump reference electrode.

The sensor element of the present invention may further include an outer sensor cell having a voltage outer electrode disposed outside the element body, the outer sensor cell being configured to generate a voltage based on an oxygen concentration in the measurement-object gas outside the element body. The flow portion pump cell may have a pump outer electrode disposed outside the element body. In this manner, the oxygen concentration in the measurement-object gas outside the element body can be detected based on the voltage of the outer sensor cell. In the sensor element, the pump outer electrode constituting part of the flow portion pump cell, and the voltage outer electrode constituting part of the outer sensor cell are both disposed outside the element body. In other words, in the sensor element, the pump outer electrode and the voltage outer electrode are separately provided outside the element body. Thus, unlike when one electrode serves as the pump outer electrode as well as the voltage outer electrode, a pump current at the time of pumping-out or pumping-in of oxygen performed by the flow portion pump cell does not flow through the voltage outer electrode, thus the voltage of the outer sensor cell does not include a voltage drop of the voltage outer electrode due to a pump current.

Consequently, the voltage of the outer sensor cell has a value which corresponds with higher accuracy to the oxygen concentration in the measurement-object gas outside the element body, thus the accuracy of detection of the oxygen concentration in the measurement-object gas using an outer sensor cell is improved.

The sensor element including the adjustment chamber pump cell according to an aspect of the present invention may further include an outer sensor cell having a voltage outer electrode disposed outside the element body, the outer sensor cell being configured to generate a voltage based on an oxygen concentration in the measurement-object gas outside the element body. The adjustment chamber pump cell may have a pump outer electrode disposed outside the element body. In other words, in an aspect in which the aforementioned outer sensor cell is provided and the flow portion pump cell has a pump outer electrode, the flow portion pump cell may be the aforementioned adjustment chamber pump cell.

In the sensor element including the outer sensor cell according to an aspect of the present invention, the outer sensor cell may have a reference electrode disposed inside the element body so as to be in contact with the reference gas serving as a reference for detecting the specific gas concentration. The reference electrode may be the aforementioned voltage reference electrode.

A first gas sensor of the present invention includes: the sensor element according to any one of the aspects described above; and a flow portion pump cell controller that causes the flow portion pump cell to pump out oxygen from the internal cavity or pump oxygen into the internal cavity by feedback-controlling the flow portion pump cell so that the voltage of the flow portion sensor cell reaches a target voltage.

In the first gas sensor, as described above, the accuracy of detection of the oxygen concentration in the internal cavity using a flow portion sensor cell of the sensor element has improved, thus the oxygen concentration in the internal cavity can be adjusted with high accuracy to an oxygen concentration corresponding to a target voltage by feedback-controlling the flow portion pump cell so that the voltage of the flow portion sensor cell reaches the target voltage.

Furthermore, in the first gas sensor, when the aforementioned pump measurement electrode and voltage measurement electrode are separately disposed in the measurement chamber of the sensor element, and the flow portion pump cell controller feedback-controls the measurement pump cell based on the voltage of the aforementioned measurement sensor cell, the specific gas concentration is detected based on the pump current which flows through the measurement pump cell by the feedback control, thus the accuracy of detection of the specific gas concentration is also improved.

In the first gas sensor of the present invention, the aforementioned flow portion pump cell controller may cause the flow portion pump cell to perform only one of pumping-out of oxygen from the internal cavity and pumping-in of oxygen to the internal cavity. For example, when the flow portion pump cell is the aforementioned measurement pump cell, the flow portion pump cell controller may cause the flow portion pump cell to perform only pumping out of oxygen from the measurement chamber.

A second gas sensor of the present invention includes: the sensor element according to an aspect in which the aforementioned adjustment chamber pump cell has a pump outer electrode; an adjustment chamber pump cell controller that causes the adjustment chamber pump cell to pump out oxygen from the oxygen concentration adjustment chamber or pump oxygen into the oxygen concentration adjustment chamber by controlling the adjustment chamber pump cell so that an oxygen concentration in the oxygen concentration adjustment chamber reaches a predetermined low concentration; and an oxygen concentration detector that detects an oxygen concentration in the measurement-object gas outside the element body based on the voltage of the outer sensor cell.

In the second gas sensor, the adjustment chamber pump cell controller controls the adjustment chamber pump cell so that the oxygen concentration in the oxygen concentration adjustment chamber reaches a predetermined low concentration. In this situation, for example, when the oxygen concentration in the measurement-object gas is switched between a high state in which the oxygen concentration is higher than a predetermined low concentration and a low state, the adjustment chamber pump cell controller switches the direction of oxygen moved by the adjustment chamber pump cell to the reverse direction. Thus, the direction of the pump current which flows through the adjustment chamber pump cell is switched to the reverse direction. Therefore, when one electrode serves as the pump outer electrode as well as the voltage outer electrode, the change in the voltage of the outer sensor cell also becomes slow due to the time required for current change when the direction of the pump current flowing through the adjustment chamber pump cell is switched to the reverse direction. In contrast, the gas sensor of the present invention is provided with the pump outer electrode and the voltage outer electrode separately, thus the voltage of the outer sensor cell is not affected by the time required for change in the pump current which flows through the adjustment chamber pump cell, and therefore, the change in the voltage of the outer sensor cell does not become slow. In other words, when the oxygen concentration in the measurement-object gas is switched between a high state in which the oxygen concentration is higher than a predetermined low concentration and a low state, the responsiveness of the voltage of the outer sensor cell is not likely to reduce.

The first or second gas sensor of the present invention may include: a reference gas adjustment unit that causes a reference-gas adjustment pump cell to perform pumping-in of oxygen to the periphery of the pump reference electrode by applying a repeatedly ON/OFF control voltage to the reference-gas adjustment pump cell; and a voltage acquisition unit that acquires the voltage of the flow portion sensor cell in a period when the repeatedly ON/OFF control voltage is OFF.

### BRIEF DESCRIPTION OF THE DRAWINGS

FIG. 1 is a schematic cross-sectional view schematically illustrating an example of the configuration of a gas sensor 100 in a first embodiment.

FIG. 2 is a top view of a pump measurement electrode 44p and a voltage measurement electrode 44s.

FIG. 3 is a block diagram illustrating an electrical connection relationship between a control device 95 and the cells of a sensor element 101.

FIG. 4 shows graphs illustrating a relationship between elapsed time and NO output change rate in an endurance test.

FIG. 5 is an explanatory chart illustrating an example of temporal change in voltage Vp3.

FIG. 6 is an explanatory chart illustrating an example of temporal change in voltage Vref.

FIG. 7 is a schematic cross-sectional view of a gas sensor 200 in a second embodiment.

FIG. 8 is a schematic cross-sectional view of a gas sensor 300 in a third embodiment.

FIG. 9 is a schematic cross-sectional view of a gas sensor 400 in a fourth embodiment.

FIG. 10 is a schematic cross-sectional view of a gas sensor 500 in a fifth embodiment.

FIG. 11 shows graphs illustrating the change in response time of voltage Vref before and after a continuous test in atmosphere.

FIG. 12 shows graphs illustrating the manner of temporal change in voltage Vref in Examples 2, 3 after a continuous test in atmosphere.

FIG. 13 is a top view of a pump measurement electrode 44p and a voltage measurement electrode 44s according to a modification.

FIG. 14 is a top view of a pump measurement electrode 44p and a voltage measurement electrode 44s according to a modification.

FIG. 15 is a partial cross-sectional view illustrating a fourth diffusion control section 60 and a third internal cavity 61 according to a modification.

FIG. 16 is a schematic cross-sectional view of a gas sensor 600 according to a modification.

FIG. 17 is a schematic cross-sectional view schematically illustrating an example of a gas sensor 900 as a conventional example.

FIG. 18 is a partial cross-sectional view illustrating a pump measurement electrode 44p and a voltage measurement electrode 44s according to a modification.

FIG. 19 is a partial cross-sectional view illustrating a pump main electrode 22p and a voltage main electrode 22s according to a modification.

### DETAILED DESCRIPTION OF THE INVENTION

### First Embodiment

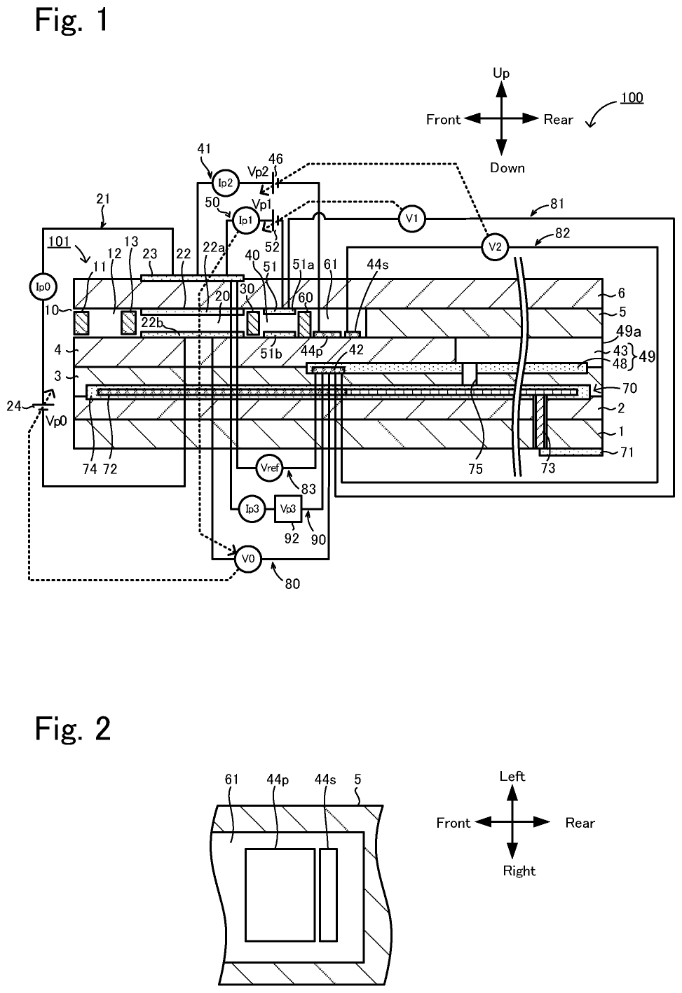

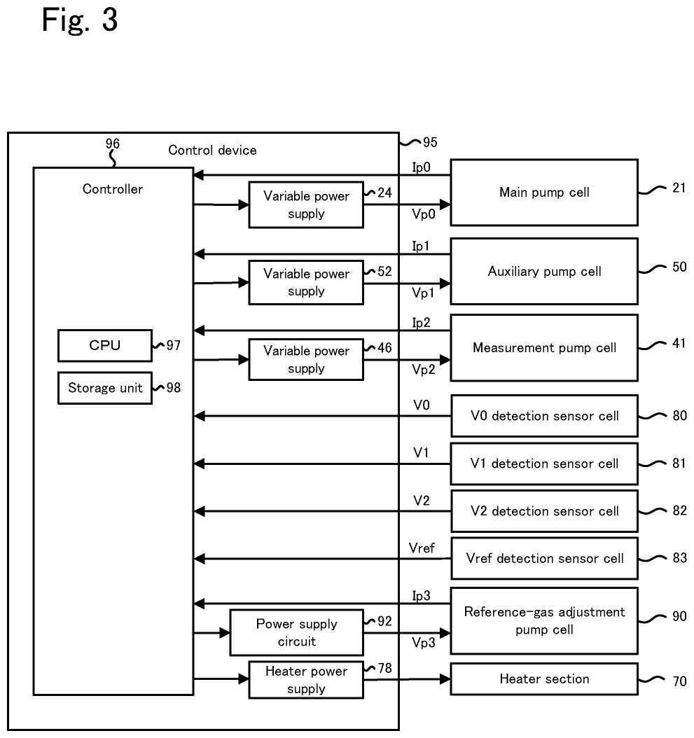

Next, an embodiment of the present invention will be described using drawings. FIG. 1 is a schematic cross-sectional view schematically illustrating an example of the configuration of a gas sensor 100 in a first embodiment of the present invention. FIG. 2 is a top view of a pump measurement electrode 44p and a voltage measurement electrode 44s of a sensor element 101. FIG. 3 is a block diagram illustrating an electrical connection relationship between a control device 95 and the cells of the sensor element 101. The gas sensor 100 includes: the sensor element 101 having an elongate rectangular parallelepiped shape; and the control device 95 that controls the entire gas sensor 100. The gas sensor 100 also includes: an element sealing body (not illustrated) that seals and fixes the sensor element 101; and a bottomed cylindrical protective cover (not illustrated) that protects the front end of the sensor element 101. The sensor element 101 includes cells 21, 41, 50, 80 to 83, 90 and a heater section 70.

The gas sensor 100 is mounted on a pipe such as the exhaust gas pipe of an internal combustion engine, for example. The gas sensor 100 detects the concentration of a specific gas such as NOx and ammonia in a measurement-object gas which is an exhaust gas of an internal combustion engine. In this embodiment, the gas sensor 100 measures the NOx concentration as the specific gas concentration. The longitudinal direction (i.e., the left-right direction in FIG. 1) of the sensor element 101 is defined as the front-rear direction, and the thickness direction (i.e., the up-down direction in FIG. 1) of the sensor element 101 is defined as the up-down direction. Furthermore, the width direction (i.e., the direction perpendicular to the front-rear direction and the up-down direction) of the sensor element 101 is defined as the left-right direction. FIG. 2 illustrates a partial cross section around the third internal cavity 61 when a spacer layer 5 is cut along the front-rear and left-right direction.

As illustrated in FIG. 1, the sensor element 101 has a layered body obtained by stacking six layers, namely, a first substrate layer 1, a second substrate layer 2, a third substrate layer 3, a first solid electrolyte layer 4, a spacer layer 5, and a second solid electrolyte layer 6 that are formed of oxygen-ion-conductive solid electrolyte layers composed of, for example, zirconia (ZrO₂), in that order from below in the drawing. The solid electrolyte used for forming each of these six layers is dense and hermetic. For example, the sensor element 101 is manufactured by performing predetermining processing and printing of a circuit pattern on ceramic green sheets corresponding to the individual layers, subsequently stacking the sheets, and then combining the sheets by calcination.

On the leading end side (front end side) of the sensor element 101 and between the lower surface of the second solid electrolyte layer 6 and the upper surface of the first solid electrolyte layer 4, a gas inlet 10, a first diffusion control section 11, a buffer space 12, a second diffusion control section 13, a first internal cavity 20, a third diffusion control section 30, a second internal cavity 40, a fourth diffusion control section 60, and a third internal cavity 61 are adjacently formed in that order to communicate with each other.

The gas inlet 10, the buffer space 12, the first internal cavity 20, the second internal cavity 40, the third internal cavity 61 form a space inside the sensor element 101, the space being provided by hollowing out the spacer layer 5 and partitioning the upper part of the space by the lower surface of the second solid electrolyte layer 6, the lower part by the upper surface of the first solid electrolyte layer 4, and the lateral part by the lateral surface of the spacer layer 5.

The first diffusion control section 11, the second diffusion control section 13, and the third diffusion control section 30 are each provided as two horizontally long slits (with an opening having a longitudinal direction in the direction perpendicular to the drawing). In addition, the fourth diffusion control section 60 is provided as one horizontally long slit (with an opening having a longitudinal direction in the direction perpendicular to the drawing) formed as a gap from the lower surface of the second solid electrolyte layer 6. Note that the portion from the gas inlet 10 to the third internal cavity 61 is also referred to as the measurement-object gas flow portion.

The sensor element 101 includes a reference-gas introduction portion 49 that causes a reference gas for measuring the NOx concentration to flow through a reference electrode 42 from the outside of the sensor element 101. The reference-gas introduction portion 49 has a reference-gas introduction space 43, and a reference-gas introduction layer 48. The reference-gas introduction space 43 is a space provided inwardly from the rear end surface of the sensor element 101. The reference-gas introduction space 43 is provided between the upper surface of the third substrate layer 3 and the lower surface of the spacer layer 5, and at the position where the lateral part is partitioned by the lateral surface of the first solid electrolyte layer 4. The reference-gas introduction space 43 has an opening in the rear end surface of the sensor element 101, and a reference gas is introduced into the reference-gas introduction space 43 through the opening. The reference-gas introduction portion 49 guides the reference gas introduced from the outside of the sensor element 101 to the reference electrode 42, while adding a predetermined diffusion resistance to the reference gas. In this embodiment, the reference gas is an atmospheric gas.

The reference-gas introduction layer 48 is provided between the upper surface of the third substrate layer 3 and the lower surface of the first solid electrolyte layer 4. The reference-gas introduction layer 48 is a porous body composed of ceramics such as alumina. Part of the upper surface of the reference-gas introduction layer 48 is exposed to the reference-gas introduction space 43. The reference-gas introduction layer 48 is formed to cover the reference electrode 42. The reference-gas introduction layer 48 causes the reference gas to flow from the reference-gas introduction space 43 to the reference electrode 42. The reference-gas introduction portion 49 does not need to include the reference-gas introduction space 43. In that case, the reference-gas introduction layer 48 should be exposed to the rear end surface of the sensor element 101.

The reference electrode 42 is interposed between the upper surface of the third substrate layer 3 and the first solid electrolyte layer 4, and as described above, in the periphery of the reference electrode 42, the reference-gas introduction layer 48 connected to the reference-gas introduction space 43 is provided. Furthermore, as will be described later, the reference electrode 42 can be used to measure the oxygen concentration (oxygen partial pressure) in the first internal cavity 20, the second internal cavity 40, and the third internal cavity 61.

The reference electrode 42 may contain a noble metal (e.g., at least one of Pt, Rh, Pd, Ru or Ir) having catalytic activity, or may be a conductive oxide sintered body containing a crystalline phase composed of a perovskite conductive oxide containing at least La, Fe and Ni. When the reference electrode 42 contains a noble metal, the reference electrode 42 is preferably composed of a cermet containing a noble metal and an oxide (in this case, ZrO₂) having oxygen ion conductivity. In addition, the reference electrode 42 is preferably a porous body. In this embodiment, the reference electrode 42 is a porous cermet electrode composed of Pt and ZrO₂.

In the measurement-object gas flow portion, the gas inlet 10 is a portion which is opened to the exterior space, and is designed to take the measurement-object gas into the sensor element 101 from the exterior space through the gas inlet 10. The first diffusion control section 11 adds a predetermined diffusion resistance to the measurement-object gas taken through the gas inlet 10. The buffer space 12 is provided to guide the measurement-object gas introduced from the first diffusion control section 11 to the second diffusion control section 13. The second diffusion control section 13 adds a predetermined diffusion resistance to the measurement-object gas introduced from the buffer space 12 into the first internal cavity 20. When the measurement-object gas is introduced from the outside of the sensor element 101 into the first internal cavity 20, the measurement-object gas suddenly taken into the sensor element 101 through the gas inlet 10 by a pressure variation (pulsation of the exhaust gas pressure when the measurement-object gas is exhaust gas of an automobile) of the measurement-object gas in the exterior space is not directly introduced into the first internal cavity 20, but is introduced into the first internal cavity 20 after the pressure variation in the measurement-object gas is cancelled through the first diffusion control section 11, the buffer space 12, and the second diffusion control section 13. Consequently, the pressure variation in the measurement-object gas introduced into the first internal cavity 20 is almost negligible. The first internal cavity 20 is provided as a space to adjust the oxygen partial pressure in the measurement-object gas introduced through the second diffusion control section 13. The oxygen partial pressure is adjusted by the main pump cell 21 operating.

The main pump cell 21 is an electrochemical pump cell including: an inner pump electrode 22 having a ceiling electrode portion 22a provided on substantially the entire lower surface of the second solid electrolyte layer 6 facing the first internal cavity 20; an outer pump electrode 23 provided to be exposed to the exterior space in an area corresponding to the ceiling electrode portion 22a on the upper surface of the second solid electrolyte layer 6; and the second solid electrolyte layer 6 interposed by these electrodes.

The inner pump electrode 22 is formed across the upper and lower solid electrolyte layers (the second solid electrolyte layer 6 and the first solid electrolyte layer 4) defining the first internal cavity 20, and the spacer layer 5 that provides a sidewall. Specifically, the ceiling electrode portion 22a is formed on the lower surface of the second solid electrolyte layer 6 providing the ceiling surface of the first internal cavity 20, a bottom electrode portion 22b is formed on the upper surface of the first solid electrolyte layer 4 providing the bottom surface, and a lateral electrode portion (not illustrated) is formed on the lateral wall surface (inner surface) of the spacer layer 5, forming both sidewalls of the first internal cavity 20 so as to connect the ceiling electrode portion 22a and the bottom electrode portion 22b, so that these electrodes are disposed in a structure of a tunnel form at the arrangement position of the lateral electrode portion.

The inner pump electrode 22 contains a noble metal (e.g., at least one of Pt, Rh, Pd, Ru or Ir) having catalytic activity. The inner pump electrode 22 also contains a noble metal (e.g., Au) having a catalytic activity inhibition ability to inhibit the catalytic activity for a specific gas of the noble metal having catalytic activity. Thus, the inner pump electrode 22 to be in contact with the measurement-object gas has a decreased reducing ability for a specific gas (in this case, NOx) component in the measurement-object gas. The inner pump electrode 22 is preferably composed of a cermet containing a noble metal and an oxide (in this case, ZrO₂) having oxygen ion conductivity. In addition, the inner pump electrode 22 is preferably a porous body. In this embodiment, the inner pump electrode 22 is a porous cermet electrode composed of Pt containing 1% of Au and ZrO₂.

As with the inner pump electrode 22, the outer pump electrode 23 contains a noble metal having catalytic activity. As with the inner pump electrode 22, the outer pump electrode 23 may be composed of a cermet. The outer pump electrode 23 is preferably a porous body. In this embodiment, the outer pump electrode 23 is a porous cermet electrode composed of Pt and ZrO₂.

In the main pump cell 21, oxygen in the first internal cavity 20 can be pumped out to the exterior space or oxygen in the exterior space can be pumped into the first internal cavity 20 by applying a desired voltage Vp0 across the inner side pump electrode 22 and the outer pump electrode 23 to cause a pump current Ip0 to flow in a positive direction or a negative direction between the inner side pump electrode 22 and the outer pump electrode 23.

Furthermore, in order to detect the oxygen concentration (oxygen partial pressure) in an atmosphere in the first internal cavity 20, an electrochemical sensor cell, that is, a V0 detection sensor cell 80 (also referred to as an oxygen partial pressure detection sensor cell for main pump control) is formed by the inner pump electrode 22, the second solid electrolyte layer 6, the spacer layer 5, the first solid electrolyte layer 4, the third substrate layer 3, and the reference electrode 42.

The oxygen concentration (oxygen partial pressure) in the first internal cavity 20 can be found by measuring the voltage V0 in the V0 detection sensor cell 80. Furthermore, the pump current Ip0 is controlled by feedback-controlling the voltage Vp0 of a variable power supply 24 so that the voltage V0 reaches a target value. Thus, the oxygen concentration in the first internal cavity 20 can be maintained at a predetermined constant value. The voltage V0 is a voltage across the inner pump electrode 22 and the reference electrode 42.

The third diffusion control section 30 adds a predetermined diffusion resistance to the measurement-object gas whose oxygen concentration (oxygen partial pressure) is controlled by the operation of the main pump cell 21 in the first internal cavity 20, and introduces the measurement-object gas to the second internal cavity 40.

After the oxygen concentration (oxygen partial pressure) is adjusted in advance in the first internal cavity 20, the second internal cavity 40 is provided as a space for further adjusting the oxygen partial pressure, by the auxiliary pump cell 50, of the measurement-object gas introduced through the third diffusion control section 30. Therefore, the oxygen concentration in the second internal cavity 40 can be maintained at a constant level with high accuracy, thus highly accurate measurement of NOx concentration is made possible in the gas sensor 100.

The auxiliary pump cell 50 is an auxiliary electrochemical pump cell including: an auxiliary pump electrode 51 having a ceiling electrode portion 51a provided on substantially the entire lower surface of the second solid electrolyte layer 6 facing the second internal cavity 40; the outer pump electrode 23 (an appropriate electrode outside the sensor element 101 suffices without being limited to the outer pump electrode 23); and the second solid electrolyte layer 6.

The auxiliary pump electrode 51 is disposed in the second internal cavity 40 in a structure of a tunnel form as in the inner pump electrode 22 provided in the aforementioned first internal cavity 20. Specifically, the ceiling electrode portion 51a is formed for the second solid electrolyte layer 6 that provides the ceiling surface of the second internal cavity 40, the bottom electrode portion 51b is formed for the first solid electrolyte layer 4 that provides the bottom surface of the second internal cavity 40, and a lateral electrode portion (not illustrated) that connects the ceiling electrode portion 51a and the bottom electrode portion 51b is formed in each of both wall surfaces of the spacer layer 5, which provide the lateral wall of the second internal cavity 40, thereby implementing a structure of a tunnel form. Note that as in the inner pump electrode 22, the auxiliary pump electrode 51 is also formed using a material having a decreased reducing ability for NOx component in the measurement-object gas.

Specifically, the auxiliary pump electrode 51 contains a noble metal (e.g., at least one of Pt, Rh, Pd, Ru or Ir) having catalytic activity. The auxiliary pump electrode 51 also contains a noble metal (e.g., Au) having the aforementioned catalytic activity inhibition ability. The auxiliary pump electrode 51 is preferably composed of a cermet containing a noble metal and an oxide (in this case, ZrO₂) having oxygen ion conductivity. In addition, the auxiliary pump electrode 51 is preferably a porous body. In this embodiment, the auxiliary pump electrode 51 is a porous cermet electrode composed of Pt containing 1% of Au and ZrO₂.

In the auxiliary pump cell 50, oxygen in an atmosphere in the second internal cavity 40 can be pumped out to the exterior space or oxygen can be pumped from the exterior space into the second internal cavity 40 by applying a desired voltage Vp1 across the auxiliary pump electrode 51 and the outer pump electrode 23.

Furthermore, in order to control the oxygen partial pressure in an atmosphere in the second internal cavity 40, an electrochemical sensor cell, that is, a V1 detection sensor cell 81 (also referred to as an auxiliary-pump-control oxygen-partial-pressure detection sensor cell) is formed by the auxiliary pump electrode 51, the reference electrode 42, the second solid electrolyte layer 6, the spacer layer 5, the first solid electrolyte layer 4, and the third substrate layer 3.

Note that the auxiliary pump cell 50 performs pumping using a variable power supply 52 whose voltage is controlled based on the voltage V1 detected by the V1 detection sensor cell 81. Thus, the oxygen partial pressure in an atmosphere in the second internal cavity 40 is controlled at a low partial pressure which has substantially no effect on measurement of NOx. The voltage V1 is a voltage across the auxiliary pump electrode 51 and the reference electrode 42.

Along with this, the pump current Ip1 is used to control the electromotive force of the V0 detection sensor cell 80. Specifically, the pump current Ip1 is input to the V0 detection sensor cell 80 as a control signal, and the aforementioned target value of the voltage V0 is controlled so that the slope of the oxygen partial pressure in the measurement-object gas introduced from the third diffusion control section 30 into the second internal cavity 40 is controlled at a constant level all the time. When the gas sensor 100 is used as an NOx sensor, the oxygen concentration in the second internal cavity 40 is maintained at a constant value around approximately 0.001 ppm by the operation of the main pump cell 21 and the auxiliary pump cell 50.

The fourth diffusion control section 60 adds a predetermined diffusion resistance to the measurement-object gas whose oxygen concentration (oxygen partial pressure) is controlled by the operation of the auxiliary pump cell 50 in the second internal cavity 40, and introduces the measurement-object gas to the third internal cavity 61. The fourth diffusion control section 60 has a function of regulating the amount of NOx which flows into the third internal cavity 61.

After the oxygen concentration (oxygen partial pressure) is adjusted in advance in the second internal cavity 40, the third internal cavity 61 is provided as a space to perform a process related to measurement of the nitrogen oxide (NOx) concentration in the measurement-object gas on the measurement-object gas introduced through the fourth diffusion control section 60. The NOx concentration is mainly measured by the operation of the measurement pump cell 41 in the third internal cavity 61.

The measurement pump cell 41 measures the NOx concentration in the measurement-object gas in the third internal cavity 61. The measurement pump cell 41 is an electrochemical pump cell including: the pump measurement electrode 44p provided on the upper surface of the first solid electrolyte layer 4 facing the third internal cavity 61; the outer pump electrode 23; the second solid electrolyte layer 6; the spacer layer 5; and the first solid electrolyte layer 4. The pump measurement electrode 44p is a porous cermet electrode composed of a material which has a higher reducing ability for NOx component in the measurement-object gas than the reducing ability of the inner pump electrode 22. The pump measurement electrode 44p also functions as an NOx reduction catalyst to reduce the NOx present in an atmosphere in the third internal cavity 61.

In the measurement pump cell 41, the oxygen generated by decomposition of nitrogen oxide in an atmosphere in the periphery of the pump measurement electrode 44p is pumped out, and the amount of generated oxygen can be detected as the pump current Ip2.

In order to detect the oxygen partial pressure in the periphery of the pump measurement electrode 44p, an electrochemical sensor cell, that is, a V2 detection sensor cell 82 (also referred to as a measurement-pump-control oxygen-partial-pressure detection sensor cell) is formed by the first solid electrolyte layer 4, the third substrate layer 3, the voltage measurement electrode 44s, and the reference electrode 42. A variable power supply 46 is controlled based on the voltage V2 detected by the V2 detection sensor cell 82. The voltage V2 is a voltage across the voltage measurement electrode 44s and the reference electrode 42.

The measurement-object gas introduced into the second internal cavity 40 reaches the pump measurement electrode 44p in the third internal cavity 61 through the fourth diffusion control section 60 in a situation where the oxygen partial pressure is controlled. The nitrogen oxide in the measurement-object gas in the periphery of the pump measurement electrode 44p is reduced (2NO→N₂+O₂) to produce oxygen. The produced oxygen is then pumped by the measurement pump cell 41, and in this process, voltage Vp2 of the variable power supply 46 is controlled so that the voltage V2 detected by the V2 detection sensor cell 82 is constant (target value). The amount of oxygen produced in the periphery of the pump measurement electrode 44p is in proportion to the concentration of nitrogen oxide in the measurement-object gas, thus the nitrogen oxide concentration in the measurement-object gas is calculated using the pump current Ip2 in the measurement pump cell 41.

An electrochemical Vref detection sensor cell 83 is formed by the second solid electrolyte layer 6, the spacer layer 5, the first solid electrolyte layer 4, the third substrate layer 3, the outer pump electrode 23, and the reference electrode 42, and the oxygen partial pressure in the measurement-object gas outside the sensor is detectable with the voltage Vref obtained by the Vref detection sensor cell 83. The voltage Vref is a voltage across the outer pump electrode 23 and the reference electrode 42.

Furthermore, an electrochemical reference-gas adjustment pump cell 90 is formed by the second solid electrolyte layer 6, the spacer layer 5, the first solid electrolyte layer 4, the third substrate layer 3, the outer pump electrode 23, and the reference electrode 42. The reference-gas adjustment pump cell 90 pumps oxygen by flowing the pump current Ip3 using a control voltage (voltage Vp3) applied by a power supply circuit 92 connected between the outer pump electrode 23 and the reference electrode 42. Thus, the reference-gas adjustment pump cell pumps oxygen from the space around the outer pump electrode 23 into the periphery of the reference electrode 42.

In the gas sensor 100 having such a configuration, the measurement-object gas having an oxygen partial pressure always maintained at a constant low value (a value having substantially no effect on measurement of NOx) is provided to the measurement pump cell 41 by operating the main pump cell 21 and the auxiliary pump cell 50. Therefore, the NOx concentration in the measurement-object gas can be found based on the pump current Ip2 which flows by pumping-out of oxygen by the measurement pump cell 41, the oxygen being produced by reduction of NOx in amount approximately proportional to the concentration of NOx in the measurement-object gas.

Furthermore, in order to enhance oxygen ion conductivity of the solid electrolyte, the sensor element 101 includes a heater section 70 having a role of temperature adjustment for heating the sensor element 101 and maintaining its temperature. The heater section 70 includes a heater connector electrode 71, a heater 72, a through-hole 73, a heater insulation layer 74, and a pressure diffusion hole 75.

The heater connector electrode 71 is formed to be in contact with the lower surface of the first substrate layer 1. Connecting the heater connector electrode 71 and an external power supply makes it possible to supply power to the heater section 70 from the outside.

The heater 72 is an electrical resistor which is formed to be interposed vertically between the second substrate layer 2 and the third substrate layer 3. The heater 72 is coupled to the heater connector electrode 71 via the through-hole 73, generates heat by being supplied with power from the outside through the heater connector electrode 71, and heats and maintains the temperature of the solid electrolyte forming the sensor element 101.

The heater 72 is buried over the entire region from the first internal cavity 20 to the third internal cavity 61, and the entire sensor element 101 can be adjusted to a temperature at which the solid electrolyte is activated.

The heater insulation layer 74 is composed of an insulator such as alumina on the upper and lower surfaces of the heater 72. The heater insulation layer 74 is formed for the purpose of obtaining an electrical insulating property between the second substrate layer 2 and the heater 72 as well as an electrical insulating property between the third substrate layer 3 and the heater 72.

The pressure diffusion hole 75 is a section provided to penetrate the third substrate layer 3 and the reference-gas introduction layer 48 so as to communicate with the reference-gas introduction space 43, and is formed for the purpose of reducing an internal pressure rise accompanied by a temperature increase in the heater insulation layer 74.

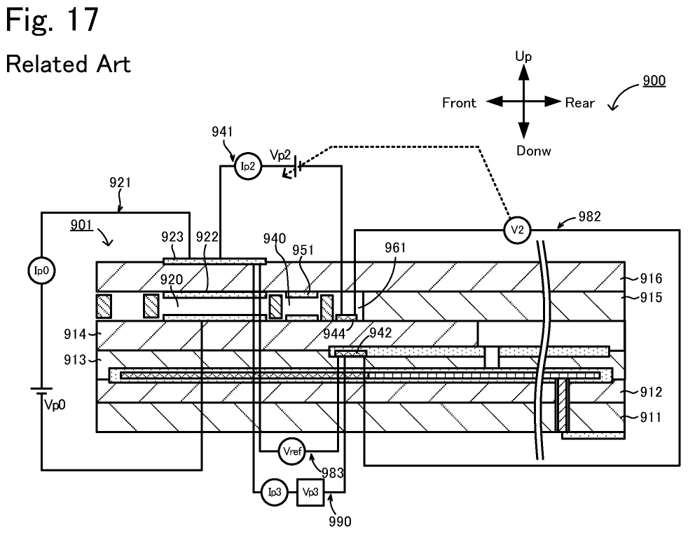

Here, the pump measurement electrode 44p and the voltage measurement electrode 44s will be described in detail. The pump measurement electrode 44p and the voltage measurement electrode 44s corresponds to an aspect in which the measurement electrode 944 in FIG. 17 is divided into two electrodes. Specifically, the measurement electrode 944 in FIG. 17 serves as the electrode of the measurement pump cell 941 to cause the pump current Ip2 to flow as well as the electrode of the measurement-pump-control oxygen-partial-pressure detection sensor cell 982 to detect the voltage V2. In contrast, in this embodiment, the pump measurement electrode 44p of the measurement pump cell 41, and the voltage measurement electrode 44s of the V2 detection sensor cell 82 are both disposed in the third internal cavity 61 as independent electrodes.

In this embodiment, as illustrated in FIG. 2, the pump measurement electrode 44p and the voltage measurement electrode 44s each have an approximately quadrangle shape in a top view. The voltage measurement electrode 44s is located rearward of the pump measurement electrode 44p. Thus, the voltage measurement electrode 44s is disposed downstream of the pump measurement electrode 44p in the measurement-object gas flow portion. The voltage measurement electrode 44s is shorter in length in the front-rear direction and smaller in area than the pump measurement electrode 44p. Note that the area of an electrode is the one as seen in the direction perpendicular to the surface where the electrode is disposed. For example, the areas of the pump measurement electrode 44p and the voltage measurement electrode 44s are each an area in a top view.

The pump measurement electrode 44p and the voltage measurement electrode 44s each contain a noble metal (e.g., at least one of Pt, Rh, Pd, Ru or Ir) having catalytic activity. In the pump measurement electrode 44p and the voltage measurement electrode 44s, the amount of contained noble metal having the aforementioned catalytic activity inhibition ability is less than the amount of the contained noble metal in the inner pump electrode 22 and the auxiliary pump electrode 51. The pump measurement electrode 44p and the voltage measurement electrode 44s preferably do not include a noble metal having the catalytic activity inhibition ability. The pump measurement electrode 44p and the voltage measurement electrode 44s are preferably composed of a cermet containing a noble metal and an oxide (in this case, ZrO₂) having oxygen ion conductivity. The pump measurement electrode 44p and the voltage measurement electrode 44s are preferably a porous body. The noble metal contained in the pump measurement electrode 44p and the noble metal contained in the voltage measurement electrode 44s may be the same in each of type and content ratio, or may be different in one of type and content ratio. It is preferable that Rh be contained in the pump measurement electrode 44p. The reaction resistance of the pump measurement electrode 44p can be reduced by containing Rh. In this embodiment, the pump measurement electrode 44p is a porous cermet electrode composed of Pt and Rh, and ZrO₂. In addition, the voltage measurement electrode 44s is a porous cermet electrode composed of Pt and ZrO₂ without containing Rh. However, the voltage measurement electrode 44s may contain Rh. For example, the mass ratio between Pt and Rh in the voltage measurement electrode 44s may be in a range of 100:0 to 30:70.

As illustrated in FIG. 3, the control device 95 includes the aforementioned variable power supplies 24, 46, 52, a heater power supply 78, the aforementioned power supply circuit 92, and a controller 96. The controller 96 is a microprocessor including a CPU 97, a RAM which is not illustrated, and a storage unit 98. The storage unit 98 is, for example, a non-volatile memory such as a ROM, which is a device that stores various data. The controller 96 receives inputs of the voltages V0 to V2 and the voltage Vref of the sensor cells 80 to 83. The controller 96 receives inputs of the pump currents Ip0 to Ip3 which flow the respective pump cells 21, 50, 41, 90. The controller 96 controls the voltages Vp0 to Vp3 output by the variable power supplies 24, 46, 52 and the power supply circuit 92 by outputting a control signal to the variable power supplies 24, 46, 52 and the power supply circuit 92, thereby controlling the pump cells 21, 41, 50, 90. The controller 96 controls the electric power to be supplied to the heater 72 by the heater power supply 78 by outputting a control signal to the heater power supply 78, thereby adjusting the temperature of the sensor element 101. The storage unit 98 stores the target value V0*, V1*, V2*, Ip1* mentioned below.

The controller 96 feedback-controls the voltage Vp0 of the variable power supply 24 so that the voltage V0 reaches a target value V0* (in other words, so that the oxygen concentration in the first internal cavity 20 reaches a target concentration).

The controller 96 feedback-controls the voltage Vp1 of the variable power supply 52 so that the voltage V1 reaches a constant value (referred to as a target value V1*) (in other words, so that the oxygen concentration in the second internal cavity 40 reaches a predetermined low oxygen concentration which has substantially no effect on measurement of NOx). Along with this, the controller 96 sets (feedback-controls) the target value V0* of the voltage V0 based on the pump current Ip1 so that the pump current Ip1 caused to flow by the voltage Vp1 reaches a constant value (referred to as a target value Ip1*). Consequently, the slope of the oxygen partial pressure in the measurement-object gas introduced from the third diffusion control section 30 into the second internal cavity 40 becomes constant all the time. In addition, the oxygen partial pressure in an atmosphere in the second internal cavity 40 is controlled at a low partial pressure which has substantially no effect on measurement of NOx. The target value V0* is set to a value that causes the oxygen concentration in the first internal cavity 20 to be higher than 0% and reach a low oxygen concentration.

The controller 96 feedback-controls the voltage Vp2 of the variable power supply 46 so that the voltage V2 reaches a constant value (referred to as a target value V2*) (in other words, the oxygen concentration in the third internal cavity 61 reaches a predetermined low concentration). Thus, oxygen is pumped out from the third internal cavity 61 so that the oxygen produced by reducing the specific gas (in this case, NOx) in the measurement-object gas in the third internal cavity 61 becomes substantially zero. The controller 96 then obtains the pump current Ip2 as a detection value corresponding to the oxygen produced from NOx in the third internal cavity 61, and calculates the NOx concentration in the measurement-object gas based on the pump current Ip2. The target value V2* is a predetermined value such that the pump current Ip2 caused to flow by the feedback-controlled voltage Vp2 becomes a limiting current. The storage unit 98 stores a relational expression (e.g., the expression of a linear function) and a map as a correspondence relationship between the pump current Ip2 and the NOx concentration. Such a relational expression and a map can be determined by an experiment in advance. The controller 96 then detects the NOx concentration in the measurement-object gas based on the obtained pump current Ip2 and the aforementioned correspondence relationship stored in the storage unit 98. In this manner, oxygen from the specific gas in the measurement-object gas introduced into the sensor element 101 is pumped out, and the specific gas concentration is detected based on the amount of oxygen pumped out (based on the pump current Ip2 in this embodiment). This method is referred to as a limiting current method.

The controller 96 causes the pump current Ip3 to flow by controlling the power supply circuit 92 so that the voltage Vp3 is applied to the reference-gas adjustment pump cell 90. The flowing of the pump current Ip3 causes the reference-gas adjustment pump cell 90 to pump in oxygen from the periphery of the outer pump electrode 23 to the periphery of the reference electrode 42.

The function of the reference-gas adjustment pump cell 90 will be described below. The measurement-object gas which has flowed into the aforementioned protective cover (not illustrated) is introduced to a measurement-object gas flow portion, such as the gas inlet 10, of the sensor element 101. In contrast, a reference gas (atmosphere) is introduced to the reference-gas introduction portion 49 of the sensor element 101. The gas inlet 10 side of the sensor element 101 and the entry side of the reference-gas introduction portion 49, in short, the front end side and the rear end side of the sensor element 101 are partitioned and sealed by the aforementioned element sealing body (not illustrated) to prevent flow of gas between the sides. However, when the pressure on the side of measurement-object gas is high, the measurement-object gas may slightly enter the reference-gas side, and the oxygen concentration of the reference gas in the periphery of the rear end side of the sensor element 101 may decrease. At this point, if the oxygen concentration in the periphery of the reference electrode 42 also decreases, the reference potential which is the electrical potential of the reference electrode 42 also changes. The voltages V0 to V2, Vref of the sensor cells 80 to 83 mentioned above are each a voltage relative to the electrical potential of the reference electrode 42, thus when the reference potential changes, the accuracy of detection of the NOx concentration in the measurement-object gas may decrease. The reference-gas adjustment pump cell 90 serves to prevent such decrease in the detection accuracy. The control device 95 controls the power supply circuit 92, and applies, as the voltage Vp3, a pulse voltage repeatedly turned ON and OFF with a predetermined cycle (e.g., 10 msec) across the reference electrode 42 and the outer pump electrode 23 of the reference-gas adjustment pump cell 90. The flowing of the pump current Ip3 through the reference-gas adjustment pump cell 90 caused by the voltage Vp3 allows oxygen to be pumped in from the periphery of the outer pump electrode 23 to the periphery of the reference electrode 42. Consequently, as described above, when the measurement-object gas causes the oxygen concentration to decrease in the periphery of the reference electrode 42, the decreased oxygen can be supplemented, and reduction in the accuracy of detection of the NOx concentration can be prevented.

Note that in addition to the variable power supplies 24, 46, 52, the heater power supply 78 and the power supply circuit 92 which are illustrated in FIG. 3, the control device 95 is actually connected to the electrodes inside the sensor element 101 through unillustrated lead wires formed in the sensor element 101, and unillustrated connector electrodes (only the heater connector electrode 71 is illustrated in FIG. 1) formed on the rear end side of the sensor element 101.

The process performed by the controller 96 at the time of detection of the NOx concentration in the measurement-object gas by the gas sensor 100 will be described. First, the CPU 97 of the controller 96 starts to drive the sensor element 101. Specifically, the CPU 97 transmits a control signal to the heater power supply 78 to heat the sensor element 101 by the heater 72. The CPU 97 then heats the sensor element 101 to a predetermined driving temperature (e.g., 800° C.). Next, the CPU 97 starts to control the aforementioned pump cells 21, 41, 50, 90, and obtain the voltages V0 to V2, Vref from the aforementioned sensor cells 80 to 83. When the measurement-object gas is introduced through the gas inlet 10 in this state, the measurement-object gas passes through the first diffusion control section 11, the buffer space 12 and the second diffusion control section 13, and reaches the first internal cavity 20. Next, the oxygen concentration of the measurement-object gas is adjusted by the main pump cell 21 and the auxiliary pump cell 50 in the first internal cavity 20 and the second internal cavity 40, and the measurement-object gas after the adjustment reaches the third internal cavity 61. The CPU 97 then detects the NOx concentration in the measurement-object gas based on the obtained pump current Ip2 and the correspondence relationship stored in the storage unit 98.

As described above, the sensor element 101 of the gas sensor 100 includes: the measurement pump cell 41 to pump out oxygen from the third internal cavity 61; and the V2 detection sensor cell 82 to generate the voltage V2 based on the oxygen concentration in the third internal cavity 61. In the third internal cavity 61, the pump measurement electrode 44p constituting part of the measurement pump cell 41, and the voltage measurement electrode 44s constituting part of the V2 detection sensor cell 82 are both disposed. In other words, in the sensor element 101 in this embodiment, the pump measurement electrode 44p and the voltage measurement electrode 44s are separately provided in the one third internal cavity 61. Thus, unlike when one electrode serves as the pump measurement electrode 44p as well as the voltage measurement electrode 44s (e.g., in the sensor element 901 illustrated in FIG. 17, the measurement electrode 944 serves as the electrode of the measurement electrode of pump cell 941 as well as the electrode of the measurement-pump-control oxygen-partial-pressure detection sensor cell 982), the pump current Ip2 at the time of pumping-out of oxygen by the measurement pump cell 41 does not flow through the voltage measurement electrode 44s. Therefore, the voltage V2 does not include a voltage drop of the voltage measurement electrode 44s due to the pump current Ip2. Thus, the voltage V2 of the V2 detection sensor cell 82 has a value which corresponds with higher accuracy to the oxygen concentration in the third internal cavity 61. More specifically, the voltage V2 has a value which corresponds with high accuracy to the electromotive force based on the oxygen concentration difference between the periphery of the voltage measurement electrode 44s and the periphery of the reference electrode 42. Therefore, the accuracy of detection the oxygen concentration in the third internal cavity 61 using the V2 detection sensor cell 82 is improved.

As described above, the voltage V2 is used to control the measurement pump cell 41, thus the NOx concentration in the measurement-object gas has more effect on the accuracy of detection of the oxygen concentration using the V2 detection sensor cell 82 than on the accuracy of detection of the oxygen concentration using the V0 detection sensor cell 80 or the V1 detection sensor cell 81. Thus, the accuracy of detection of the NOx concentration is improved by improving the accuracy of detection of the oxygen concentration in the third internal cavity 61 using the V2 detection sensor cell 82.

Note that when one measurement electrode 944 is provided as in the sensor element 901 in a conventional example, and the pump measurement electrode 44p and the voltage measurement electrode 44s are not independent, in addition to the electromotive force based on the oxygen concentration difference between the periphery of the measurement electrode 944 and the periphery of the reference electrode 942, the voltage V2 of the measurement-pump-control oxygen-partial-pressure detection sensor cell 982 includes the value (voltage drop) obtained by multiplying the pump current Ip2 of the measurement pump cell 941 by the resistance of the measurement electrode 944. Regarding the magnitude of a voltage drop in the measurement electrode 944, due to effect of a manufacturing variation (e.g., a variation in state, such as thickness, degree of porosity, surface area) of the measurement electrode 944, when multiple sensor elements 901 are manufactured, individual difference may occur for each sensor element 901. Thus, in the sensor element 901, the accuracy of detection of the oxygen concentration in the third internal cavity 961 using the voltage V2 may have a variation for each sensor element 901. In contrast, in the sensor element 101 in this embodiment, a voltage drop does not occur in the voltage measurement electrode 44s when the pump current Ip2 is not passed through the voltage measurement electrode 44s, thus even when a plurality of sensor elements 101 have a manufacturing variation in the voltage measurement electrode 44s, the accuracy of detection of the oxygen concentration in the third internal cavity 61 using the voltage V2 is unlikely to have a variation.

As described above, the controller 96 causes the measurement pump cell 41 to pump out oxygen from the third internal cavity 61 by feedback-controlling the measurement pump cell 41 so that the voltage V2 of the V2 detection sensor cell 82 reaches a target voltage (target value V2*). As described above, in the sensor element 101 in this embodiment, the accuracy of detection of the oxygen concentration in the third internal cavity 61 using the V2 detection sensor cell 82 has been improved, thus the oxygen concentration in the third internal cavity 61 can be adjusted with high accuracy to the oxygen concentration corresponding to the target value V2* by performing the aforementioned feedback control so that the voltage V2 reaches the target value V2*. In addition, the NOx concentration is detected based on the pump current Ip2 which flows through the measurement pump cell 41 by this feedback control, thus the accuracy of detection of the NOx concentration is also improved.

Disposing the pump measurement electrode 44p and the voltage measurement electrode 44s separately can prevent reduction (hereinafter referred to as “deterioration of the accuracy of detection”) in the accuracy of detection of the NOx concentration with use of the gas sensor 100. The reason for this will be described. As illustrated in FIG. 17, in the sensor element 901 in a conventional example, the pump measurement electrode 44p and the voltage measurement electrode 44s are not separated, but one measurement electrode 944 is disposed instead. In this case, as described above, in addition to the electromotive force based on the oxygen concentration difference between the periphery of the measurement electrode 944 and the periphery of the reference electrode 942, the voltage V2 includes a voltage drop in the measurement electrode 944 due to the pump current Ip2. Thus, when the measurement pump cell 941 is controlled so that the voltage V2 reaches the target value V2*, the greater the voltage drop, the lower the electromotive force. In other words, even when the same control is performed for the measurement pump cell 941, the greater the voltage drop, the smaller the oxygen concentration difference between the periphery of the measurement electrode 944 and the periphery of the reference electrode 942, thus the oxygen concentration in the periphery of the measurement electrode 944 approaches the oxygen concentration of the reference gas. In other words, the oxygen concentration in the periphery of the measurement electrode 944 becomes higher than a target low concentration. Meanwhile, the noble metal in the measurement electrode 944 may be oxidized by flowing the pump current Ip2. For example, when Pt and Rh are contained in the measurement electrode 944, part of these may be oxidized to produce PtO, PtO₂, and Rh₂O₃. Such oxidation of a noble metal is likely to occur, particularly when the oxygen concentration in the periphery of the measurement electrode 944 is high. Oxidized noble metal is more likely to be evaporated than the noble metal before being oxidized, thus the noble metal in the measurement electrode 944 decreases with use of the gas sensor 900, and the catalytic activity of the measurement electrode 944 is reduced. In short, the measurement electrode 944 deteriorates. When the catalytic activity of the measurement electrode 944 is reduced, the reaction resistance of the measurement electrode 944 increases. In addition, as the reaction resistance of the measurement electrode 944 increases, the voltage drop is further increased, thus when the measurement pump cell 941 is controlled based on the voltage V2, the oxygen concentration in the periphery of the measurement electrode 944 is further increased, and the measurement electrode 944 further deteriorates and the reaction resistance increases. When the reaction resistance of the measurement electrode 944 increases, the pump current Ip2 cannot reach a limiting current, and the pump current Ip2 decreases, thus the pump current Ip2 deviates from a correct value corresponding to the NOx concentration, and therefore, the accuracy of detection of the NOx concentration decreases. For this reason, the accuracy of detection of the NOx concentration decreases with use of the gas sensor 900 of FIG. 17. In contrast, in this embodiment, the pump current Ip2 is not passed through the voltage measurement electrode 44s, thus the voltage measurement electrode 44s is unlikely to deteriorate. Even if the voltage measurement electrode 44s deteriorates, the pump current Ip2 is not passed therethrough, thus a voltage drop does not occur. Because of this, even when the gas sensor 100 is used for a long time, the accuracy of detection of the oxygen concentration in the third internal cavity 61 using the voltage V2 is unlikely to decrease, thus even when the gas sensor 100 is used for a long time, the oxygen concentration in the periphery of the pump measurement electrode 44p is unlikely to increase. Therefore, deterioration (reduction in catalytic activity) of the pump measurement electrode 44p is prevented, and deterioration of the accuracy of detection of the NOx concentration is prevented.

Note that in addition to the aforementioned electromotive force based on the oxygen concentration difference between the periphery of the voltage electrode 44s and the periphery of the reference electrode 42, the voltage V2 also includes the thermal electromotive force of the voltage measurement electrode 44s. Thus, in order to further improve the accuracy of detection of the oxygen concentration using the V2 detection sensor cell 82, it is preferable to reduce the thermal electromotive force of the voltage measurement electrode 44s. The aforementioned deterioration of the pump measurement electrode 44p is further prevented by reducing the thermal electromotive force of the voltage measurement electrode 44s, thus deterioration of the accuracy of detection of the NOx concentration is also further prevented. For example, a temperature variation in the voltage measurement electrode 44s can be reduced by decreasing the area of the voltage measurement electrode 44s as much as possible, thus the thermal electromotive force of the voltage measurement electrode 44s can be reduced. The voltage measurement electrode 44s may have a high resistance value because the pump current Ip2 does not flow therethrough, thus is more easily reduced in area than the pump measurement electrode 44p. In this embodiment, as described above, the area of the voltage measurement electrode 44s is made smaller than the area of the pump measurement electrode 44p, thus the thermal electromotive force of the voltage measurement electrode 44s can be made relatively small.

The pump measurement electrode 44p and the voltage measurement electrode 44s are preferably disposed as close as possible in a range where both are not in contact with each other (not conductive to each other). In this manner, the voltage V2 measured using the voltage measurement electrode 44s has a value which corresponds with higher accuracy to the oxygen concentration in the periphery of the pump measurement electrode 44p, thus the accuracy of measurement of the NOx concentration is improved. In this embodiment, as illustrated in FIG. 2, the pump measurement electrode 44p and the voltage measurement electrode 44s are adjacent in the front-rear direction so that both are disposed as close as possible.

As illustrated in FIG. 2, the voltage measurement electrode 44s is preferably disposed downstream of the measurement-object gas relative to the pump measurement electrode 44p. In this manner, the oxygen concentration in the measurement-object gas after pumping out oxygen in the periphery of the pump measurement electrode 44p using the pump current Ip2 can be detected based on the voltage V2. Thus, as described above, when the measurement pump cell 41 is feedback-controlled so that the voltage V2 reaches the target value V2*, the oxygen concentration in the third internal cavity 61 can be adjusted with high accuracy to the oxygen concentration corresponding to the target value V2*.

The manner of the aforementioned change in the accuracy of detection of the NOx concentration with use of the gas sensor 100 has been studied in the following way. First, Example 1 is implemented by producing the sensor element 101 and the gas sensor 100 in this embodiment illustrated in FIGS. 1 to 3. The area ratio between the pump measurement electrode 44p and the voltage measurement electrode 44s is 5:1. In addition, Comparative Example 1 is implemented by producing a gas sensor which is the same as Example 1 except that the pump measurement electrode 44p and the voltage measurement electrode 44s are not included but the measurement electrode 944 of FIG. 17 is included instead. In Comparative Example 1, the measurement electrode 944 constitutes part of each of the measurement pump cell 41 and the V2 detection sensor cell 82. The same material is used for the pump measurement electrode 44p in Example 1 and the measurement electrode 944 of Comparative Example 1. The voltage measurement electrode 44s in Example 1 uses the same material as for the pump measurement electrode 44p except that Rh is not contained.

An endurance test using a diesel engine was conducted for Example 1 and Comparative Example 1 to evaluate the degree of deterioration of the accuracy of detection of the NOx concentration. First, the gas sensor in Example 1 was mounted on a model gas device. The heater 72 was energized to attain a temperature of 800° C. to heat the sensor element 101. A state is achieved in which the aforementioned pump cells 21, 41, 50 are controlled by the controller 96, and the voltages V0, V1, V2, Vref are obtained from the aforementioned sensor cells 80 to 83. A state is achieved in which the reference-gas adjustment pump cell 90 is not controlled by the controller 96. In this state, a first model gas having a base gas of nitrogen and an NO concentration of 1500 ppm is passed through a model gas device, and the standby state is maintained until the pump current Ip2 is stabilized. The pump current Ip2 after stabilized was measured as an initial value Ia of the output of the gas sensor for NO. Subsequently, an endurance test was conducted as follows. First, the gas sensor in Example 1 was mounted on the exhaust gas pipe of an automobile. Then, a 40-minute operation pattern constructed by an engine rotation speed in a range of 1500 to 3500 rpm and a load torque in a range of 0 to 350 N·m was repeated until 500 hours have elapsed. Note that the gas temperature then was 200° C. to 600° C., and the NOx concentration was 0 to 1500 ppm. The controller 96 continued to control the aforementioned pump cells and obtain the voltages during the 500 hours. After lapse of 500 hours, the gas sensor is temporarily removed from the exhaust gas pipe and is mounted on the model gas device, and the value of the pump current Ip2 was measured by the same method as for the initial value Ia to obtain value Ib after lapse of 500 hours. NO output change rate [%] of the pump current Ip2 of the gas sensor in Example 1 after lapse of 500 hours was derived from NO output change rate after lapse of 500 hours=[1−(Ib/Ia)]×100%. Similarly, 500-hour endurance test and subsequent measurement of the value Ib were repeatedly conducted, and NO output change rate was derived for the total elapsed time of the endurance test of each of 1000 hours, 1500 hours, 2000 hours, 2500 hours, and 3000 hours. For the gas sensor of Comparative Example 1, similarly, NO output change rate was derived for the initial value Ia and the elapsed time of the endurance test up to 3000 hours.

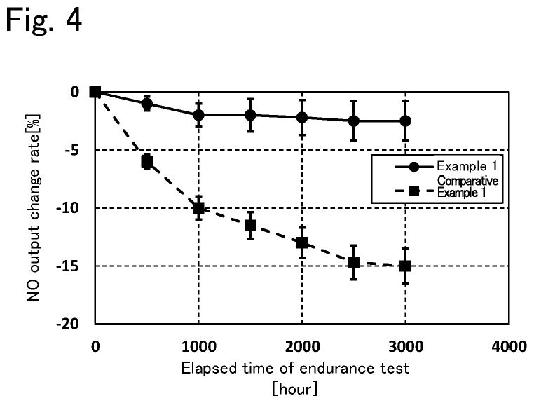

FIG. 4 shows graphs illustrating a relationship between elapsed time and NO output change rate in the aforementioned endurance test in Example 1 and Comparative Example 1. In each of Example 1 and Comparative Example 1, NO output change rate is shown, where the initial value Ia for the elapsed time of 0 hour is used as a reference (=NO output change rate is 0%). The smaller the absolute value of NO output change rate, the lower the change in the pump current Ip2 for NO after an endurance test, which shows that deterioration of the accuracy of detection of the NOx concentration is prevented. Note that FIG. 4 shows the result of the aforementioned endurance test for five gas sensors in each of Example 1 and Comparative Example 1, and illustrates the average for five gas sensors as the value of NO output change rate. In addition, in FIG. 4, for NO output change rate for the total elapsed time of 500 hours to 3000 hours of the endurance test, a maximum value and a minimum value among five gas sensors are also illustrated. As illustrated in FIG. 4, as compared to Comparative Example 1 in which the measurement electrode 944 is disposed instead of these electrodes, deterioration of the accuracy of detection of the NOx concentration is further prevented in Example 1 in which the pump measurement electrode 44p and the voltage measurement electrode 44s are both disposed. This is probably because when the endurance test is conducted, deterioration of the pump measurement electrode 44p in Example 1 is more prevented than the measurement electrode 944 of Comparative Example 1 due to the aforementioned reason.

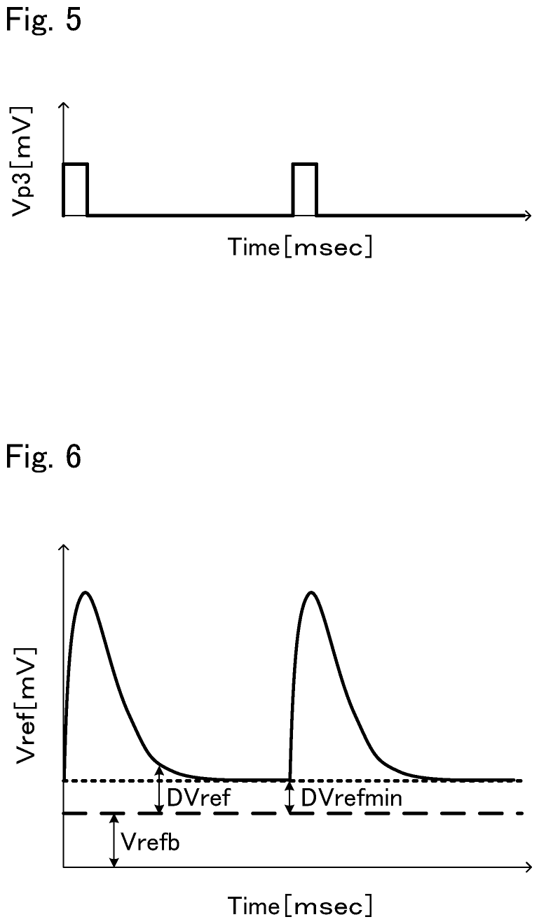

Note that in addition to the aforementioned electromotive force based on the oxygen concentration difference between the periphery of the voltage measurement electrode 44s and the periphery of the reference electrode 42, and the thermal electromotive force of the voltage measurement electrode 44s, the voltage V2 includes the value (voltage drop) obtained by multiplying the pump current Ip3 of the reference-gas adjustment pump cell 90 by the resistance of the reference electrode 42. In other words, the reference potential that is the electrical potential of the reference electrode 42 changes due to the magnitude of a voltage drop thereof occurred according to the pump current Ip3 that flows through the reference electrode 42, and thus the voltage V2 also changes. This will be described. FIG. 5 is an explanatory chart illustrating an example of temporal change in the voltage Vp3. FIG. 6 is an explanatory chart illustrating an example of temporal change in the voltage Vref. When the pulse voltage of FIG. 5 is applied across the reference electrode 42 and the outer pump electrode 23 as the voltage Vp3, the voltage Vref across the reference electrode 42 and the outer pump electrode 23 varies like the waveform of FIG. 6. Specifically, when the pulse voltage of the voltage Vp3 is turned ON, the voltage Vref gradually rises accordingly, while when the pulse voltage of the voltage Vp3 is turned OFF, the voltage Vref gradually falls accordingly, and the voltage Vref has a minimum value immediately before the pulse voltage is turned ON subsequently. The reason why the voltage Vref varies in this manner is that the voltage Vref includes a voltage drop caused by the pump current Ip3 that flows through the reference electrode 42. Specifically, rise and fall of the pump current Ip3 is repeated due to the pulse voltage as in the waveform in FIG. 6, thus the magnitude of the voltage drop of the reference electrode 42 also varies according to the pump current Ip3, and the voltage Vref varies like the waveform in FIG. 6. In FIG. 6, the original value (the voltage based on the oxygen concentration difference between the periphery of the reference electrode 42 and the periphery of the outer pump electrode 23) of the voltage Vref is shown as base voltage Vrefb. Residual voltage DVref that is the difference between the voltage Vref and the base voltage Vrefb includes a voltage drop of the reference electrode 42. The lower the residual voltage DVref, the smaller the change in the electrical potential of the reference electrode 42 due to the pump current Ip3, and the smaller the change in the voltage V2 caused by the change in the electrical potential of the reference electrode 42. Thus, the controller 96 preferably obtains the voltage V2 in a period when the voltage Vp3 is OFF, and more preferably, obtains the voltage V2 at a timing with the residual voltage DVref as low as possible in the OFF-period of the voltage Vp3. In this manner, reduction in the accuracy of measurement of the oxygen concentration in the third internal cavity 61, caused by the pump current Ip3 can be prevented and the voltage V2 has a value which corresponds with higher accuracy to the oxygen concentration in the third internal cavity 61. In addition, when the controller 96 feedback-controls the measurement pump cell 41 based on the voltage V2 obtained at such timing, the oxygen concentration in the third internal cavity 61 can be adjusted with high accuracy to the oxygen concentration corresponding to the target value V2*.

Specifically, the timing with the residual voltage DVref as low as possible may be any timing in the following period. Specifically, first, in one cycle of ON and OFF of the voltage Vp3, the maximum of the value of the voltage Vref is assumed to be 100%, and the minimum is assumed to be 0%. Let the period with a low residual voltage DVref be the period since the voltage Vref falls below 10% after turn-OFF of the voltage Vp3 until the voltage Vref starts to rise due to turn-ON of the voltage Vp3 in the next cycle. The controller 96 preferably obtains the voltage V2 at any timing in this period. More preferably, the controller 96 obtains the voltage V2 at the timing of a minimum DVrefmin (see FIG. 6) of the residual voltage DVref in one cycle of ON and OFF of the voltage Vp3. When the voltage Vref is stable in an OFF-period of the voltage Vp3 (until the voltage Vp3 is turned ON subsequently) as in the waveform of FIG. 6, the controller 96 may obtain the voltage V2 at any timing in the period in which the voltage Vref is stable. In this manner, the controller 96 can obtain the voltage V2 at the timing when the residual voltage DVref attains the minimum DVrefmin. In contrast, when the voltage Vref is unstable in an OFF-period of the voltage Vp3, the residual voltage DVref attains the minimum DVrefmin at the timing immediately before the subsequent turn-ON in the OFF-period of the voltage Vp3, thus the controller 96 preferably obtains the voltage V2 at this timing. The timing when the controller 96 preferably obtains the voltage V2 can be determined in advance by an experiment based on the ON/OFF cycle of the voltage Vp3, the pump current Ip3 and the waveform of temporal change in the voltage Vref caused by the voltage Vp3.

Note that for the sake of explanation, FIG. 6 illustrates the waveform of the voltage Vref when the base voltage Vrefb is constant, specifically, when the oxygen concentration in the measurement-object gas in the periphery of the outer pump electrode 23 is constant. Actually, the base voltage Vrefb varies according to the oxygen concentration in the measurement-object gas in the periphery of the outer pump electrode 23, thus the voltage Vref also changes due to the variation in the base voltage Vrefb.

As with the voltage V2, the voltages V0, V1, Vref are affected by the pump current Ip3. Thus, as with the voltage V2, the controller 96 obtains the voltages V0, V1, Vref preferably in an OFF-period of the voltage Vp3, more preferably in the aforementioned period with a low residual voltage DVref, and still more preferably at any timing in the period in which the voltage Vref is stable or at the timing immediately before the subsequent turn-ON in an OFF-period. In addition, as with the voltage V2, the controller 96 obtains the pump currents Ip0 to Ip3 preferably in an OFF-period of the voltage Vp3, more preferably in the aforementioned period with a low residual voltage DVref, and still more preferably at any timing in the period in which the voltage Vref is stable or at the timing immediately before the subsequent turn-ON in an OFF-period. In this embodiment, the controller 96 obtains the voltages V0, V1, V2, Vref, and the pump currents Ip0 to Ip3 at the timing immediately before the subsequent turn-ON in an OFF-period of the voltage Vp3.

The correspondence relationships between the components in this embodiment and the components in the present invention will now be clarified. The first substrate layer 1, the second substrate layer 2, the third substrate layer 3, the first solid electrolyte layer 4, the spacer layer 5 and the second solid electrolyte layer 6 correspond to an element body according to the present invention, the third internal cavity 61 corresponds to an internal cavity and a measurement chamber, the pump measurement electrode 44p corresponds to a pump inner electrode and a pump measurement electrode, the measurement pump cell 41 corresponds to a flow portion pump cell and a measurement pump cell, the voltage measurement electrode 44s corresponds to a voltage inner electrode and a voltage measurement electrode, and the V2 detection sensor cell 82 corresponds to a flow portion sensor cell and a measurement sensor cell. In addition, the first internal cavity 20 and the second internal cavity 40 corresponds to an oxygen concentration adjustment chamber, and the main pump cell 21 and the auxiliary pump cell 50 correspond to an adjustment chamber pump cell. The controller 96 corresponds to a flow portion pump cell controller. The outer pump electrode 23 corresponds to a pump electrode and a pump outer electrode of a flow portion pump cell. The reference electrode 42 corresponds to a reference electrode.

According to the gas sensor 100 in this embodiment described above in detail, in the sensor element 101, the pump measurement electrode 44p and the voltage measurement electrode 44s are separately provided in one third internal cavity 61. Thus, the voltage V2 does not include a voltage drop of the voltage measurement electrode 44s due to the pump current Ip2. Therefore, the accuracy of detection of the oxygen concentration in the third internal cavity 61 using the V2 detection sensor cell 82 is improved. The voltage V2 is used to control the measurement pump cell 41, thus has a greater effect on the accuracy of detection of the NOx concentration in the measurement-object gas than the voltages V0, V1, for example. Thus, the accuracy of detection of the NOx concentration is improved by improving the accuracy of detection of the oxygen concentration in the third internal cavity 61 using the V2 detection sensor cell 82.

Furthermore, the controller 96 causes the measurement pump cell 41 to pump out oxygen from the third internal cavity 61 by feedback-controlling the measurement pump cell 41 so that the voltage V2 reaches the target value V2*. As described above, the accuracy of detection of the oxygen concentration in the third internal cavity 61 using the V2 detection sensor cell 82 of the sensor element 101 is improved by separately disposing the pump measurement electrode 44p and the voltage measurement electrode 44s, thus the oxygen concentration in the third internal cavity 61 can be adjusted with high accuracy to the oxygen concentration corresponding to the target value V2* by performing the aforementioned feedback control. In addition, the NOx concentration is detected by this feedback control based on the pump current Ip2 which flows through the measurement pump cell 41, thus the accuracy of detection of the NOx concentration is also improved.

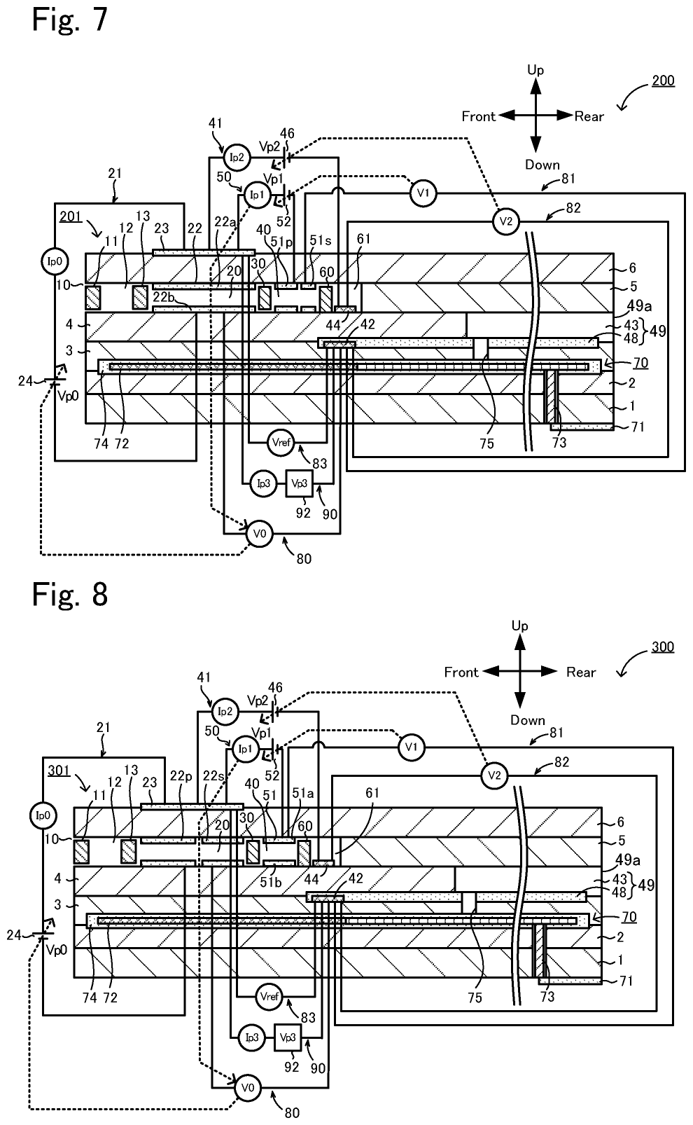

In the embodiment described above, the pump measurement electrode 44p and the voltage measurement electrode 44s are both provided in the third internal cavity 61, but are not limited thereto. An aspect may be provided in which regarding the electrodes disposed in internal cavities of the measurement-object gas flow portion, the pump measurement electrode and the voltage measurement electrode are separately provided in the same internal cavity. For example, instead of the auxiliary pump electrode 51 in FIG. 1, a pump auxiliary electrode 51p and a voltage auxiliary electrode 51s may be disposed in the second internal cavity 40 as illustrated in FIG. 7. This case will be described in the second embodiment explained later. Alternatively, instead of the inner pump electrode 22 of FIG. 1, a pump main electrode 22p and a voltage main electrode 22s may be disposed in the first internal cavity as illustrated in FIG. 8. This case will be described in the third embodiment explained later.

### Second Embodiment

FIG. 7 is a schematic cross-sectional view schematically illustrating an example of the configuration of a gas sensor 200 in a second embodiment. A sensor element 201 of the gas sensor 200 includes the pump auxiliary electrode 51p and the voltage auxiliary electrode 51s instead of the auxiliary pump electrode 51 in FIG. 1. In addition, the sensor element 201 includes one measurement electrode 44 instead of the pump measurement electrode 44p and the voltage measurement electrode 44s in FIG. 1. The measurement electrode 44 serves as the electrode of the measurement pump cell 41 as well as the electrode of the V2 detection sensor cell 82. The pump auxiliary electrode 51p constitutes part of the auxiliary pump cell 50, and the pump current Ip1 flows through the pump auxiliary electrode 51p. The voltage auxiliary electrode 51s constitutes part of the V1 detection sensor cell 81, and the voltage across the voltage auxiliary electrode 51s and the reference electrode 42 gives voltage V1. As with the auxiliary pump electrode 51, the pump auxiliary electrode 51p and the voltage auxiliary electrode 51s each have a structure in a tunnel form. The voltage auxiliary electrode 51s is disposed downstream of the pump auxiliary electrode 51p in the measurement-object gas flow portion. The voltage auxiliary electrode 51s is shorter in length in the front-rear direction than the pump auxiliary electrode 51p, and accordingly, the area of the voltage auxiliary electrode 51s is smaller than the area of the pump auxiliary electrode 51p. The material for the pump auxiliary electrode 51p and the voltage auxiliary electrode 51s is the same as for the auxiliary pump electrode 51 in the first embodiment. However, the noble metal contained in the pump measurement electrode 51p and the noble metal contained in the voltage auxiliary electrode 51s may be different in at least one of type or content ratio.

Except this point, the gas sensor 200 is the same as the gas sensor 100 in the first embodiment. For example, as in the first embodiment, the controller 96 feedback-controls the voltage Vp1 of the variable power supply 52 so that the voltage V1 reaches the target value V1*, thus the pump current Ip1 flows through the auxiliary pump cell 50.

Of the correspondence relationships between the components in this embodiment and the components in the present invention, particularly, the correspondence relationships different from those in the first embodiment will now be clarified. The second internal cavity 40 in this embodiment corresponds to an internal cavity, an oxygen concentration adjustment chamber and a second internal cavity, the pump auxiliary electrode 51p corresponds to a pump inner electrode, a pump adjustment electrode and a pump auxiliary electrode, the auxiliary pump cell 50 corresponds to a flow portion pump cell, an adjustment chamber pump cell and an auxiliary pump cell, the voltage auxiliary electrode 51s corresponds to a voltage inner electrode, a voltage adjustment electrode and a voltage auxiliary electrode, and the V1 detection sensor cell 81 corresponds to a flow portion sensor cell, an adjustment chamber sensor cell and a second internal cavity sensor cell. In addition, the third internal cavity 61 corresponds to a measurement chamber, and the controller 96 corresponds to a flow portion pump cell controller. The outer pump electrode 23 corresponds to a pump electrode and a pump outer electrode of the flow portion pump cell.

In the gas sensor 200 in this embodiment described above in detail, in the sensor element 201, the pump auxiliary electrode 51p and the voltage auxiliary electrode 51s are separately provided in one second internal cavity 40. Thus, the same effect as the one achieved by separately providing the pump measurement electrode 44p and the voltage measurement electrode 44s in the above-described first embodiment is obtained. For example, since the pump current Ip1 does not flow through the voltage auxiliary electrode 51s, the voltage V1 does not include a voltage drop of the voltage auxiliary electrode 51s due to the pump current Ip1. Thus, the voltage V1 of the V1 detection sensor cell 81 has a value which corresponds with higher accuracy to the oxygen concentration in the second internal cavity 40. More specifically, the voltage V1 has a value which corresponds with high accuracy to the electromotive force based on the oxygen concentration difference between the periphery of the voltage auxiliary electrode 51s and the periphery of the reference electrode 42. Therefore, the accuracy of detection the oxygen concentration in the second internal cavity 40 using the V1 detection sensor cell 81 is improved. In addition, even when a plurality of sensor elements 201 have a manufacturing variation in the voltage auxiliary electrode 51s, the accuracy of detection of the oxygen concentration in the second internal cavity 40 using the voltage V1 is unlikely to have a variation.

The controller 96 causes the auxiliary pump cell 50 to pump out oxygen from the second internal cavity 40 or pump oxygen into the second internal cavity 40 by feedback-controlling the auxiliary pump cell 50 so that the voltage V1 reaches to target value V1*. Thus, the oxygen concentration in the second internal cavity 40 can be adjusted with high accuracy to the oxygen concentration corresponding to the target value V1*. In addition, even when the sensor element 201 is used for a long time, the accuracy of detection of the oxygen concentration in the second internal cavity 40 with the voltage V1 is unlikely to decrease, thus even when the sensor element 201 is used for a long time, the oxygen concentration in the periphery of the pump auxiliary electrode 51p is unlikely to increase. Therefore, deterioration (reduction in catalytic activity) of the pump auxiliary electrode 51p is prevented.

### Third Embodiment

FIG. 8 is a schematic cross-sectional view schematically illustrating an example of the configuration of a gas sensor 300 in a third embodiment. A sensor element 301 of the gas sensor 300 includes a pump main electrode 22p and a voltage main electrode 22s instead of the inner pump electrode 22 in FIG. 1. In addition, as with the sensor element 201, the sensor element 301 includes one measurement electrode 44 instead of the pump measurement electrode 44p and the voltage measurement electrode 44s in FIG. 1. The pump main electrode 22p constitutes part of the main pump cell 21, and the pump current Ip0 flows through the pump main electrode 22p. The voltage main electrode 22s constitutes part of the V0 detection sensor cell 80, and the voltage across the voltage main electrode 22s and the reference electrode 42 gives voltage V0. As with the inner pump electrode 22, the pump main electrode 22p and the voltage main electrode 22s each have a structure in a tunnel form. The voltage main electrode 22s is disposed downstream of the pump main electrode 22p in the measurement-object gas flow portion. The voltage main electrode 22s is shorter in length in the front-rear direction than the pump main electrode 22p, and accordingly, the area of the voltage main electrode 22s is smaller than the area of the pump main electrode 22p. The material for the pump main electrode 22p and the voltage main electrode 22s is the same as for the inner pump electrode 22 in the first embodiment. However, the noble metal contained in the pump main electrode 22p and the noble metal contained in the voltage main electrode 22s may be different in at least one of type or content ratio.

Except this point, the gas sensor 300 is the same as the gas sensor 100 in the first embodiment. For example, as in the first embodiment, the controller 96 feedback-controls the voltage Vp0 of the variable power supply 24 so that the voltage V0 reaches the target value V0*, thus the pump current Ip0 flows through the main pump cell 21.

Of the correspondence relationships between the components in this embodiment and the components in the present invention, particularly, the correspondence relationships different from those in the first embodiment will now be clarified. The first internal cavity 20 in this embodiment corresponds to an internal cavity, an oxygen concentration adjustment chamber and a first internal cavity, the pump main electrode 22p corresponds to a pump inner electrode, a pump adjustment electrode and a pump main electrode, the main pump cell 21 corresponds to a flow portion pump cell, an adjustment chamber pump cell and a main pump cell, the voltage main electrode 22s corresponds to a voltage inner electrode, a voltage adjustment electrode and a voltage main electrode, and the V0 detection sensor cell 80 corresponds to a flow portion sensor cell, an adjustment chamber sensor cell and a first internal cavity sensor cell. In addition, the third internal cavity 61 corresponds to a measurement chamber, and the controller 96 corresponds to a flow portion pump cell controller. The outer pump electrode 23 corresponds to a pump electrode and a pump outer electrode of the flow portion pump cell.

In the gas sensor 300 in this embodiment described above in detail, in the sensor element 301, the pump main electrode 22p and the voltage main electrode 22s are separately provided in one first internal cavity 20. Thus, the same effect as the one achieved by separately providing the pump measurement electrode 44p and the voltage measurement electrode 44s in the above-described first embodiment is obtained. For example, since the pump current Ip0 does not flow through the voltage main electrode 22s, the voltage V0 does not include a voltage drop of the voltage main electrode 22s due to the pump current Ip0. Thus, the voltage V0 of the V0 detection sensor cell 80 has a value which corresponds with higher accuracy to the oxygen concentration in the first internal cavity 20. More specifically, the voltage V0 has a value which corresponds with higher accuracy to the electromotive force based on the oxygen concentration difference between the periphery of the voltage main electrode 22s and the periphery of the reference electrode 42. Therefore, the accuracy of detection the oxygen concentration in the first internal cavity 20 using the V0 detection sensor cell 80 is improved. In addition, even when a plurality of sensor elements 301 have a manufacturing variation in the voltage main electrode 22s, the accuracy of detection of the oxygen concentration in the first internal cavity 20 with the voltage V0 is unlikely to have a variation.

The controller 96 causes the main pump cell 21 to pump out oxygen from the first internal cavity 20 or pump oxygen into the first internal cavity 20 by feedback-controlling the main pump cell 21 so that the voltage V0 reaches to the target value V0*. Thus, the oxygen concentration in the first internal cavity 20 can be adjusted with high accuracy to the oxygen concentration corresponding to the target value V0*. In addition, even when the sensor element 301 is used for a long time, the accuracy of detection of the oxygen concentration in the first internal cavity 20 with the voltage V0 is unlikely to decrease, thus even when the sensor element 301 is used for a long time, the oxygen concentration in the periphery of the pump main electrode 22p is unlikely to increase. Therefore, deterioration (reduction in catalytic activity) of the pump main electrode 22p is prevented.

### Fourth Embodiment

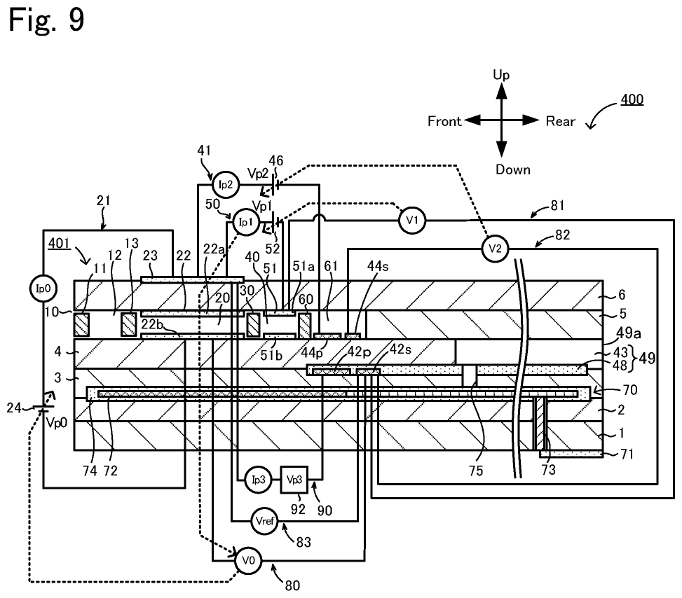

FIG. 9 is a schematic cross-sectional view schematically illustrating an example of the configuration of a gas sensor 400 in a fourth embodiment. As with the sensor element 101, a sensor element 401 of the gas sensor 400 includes the pump measurement electrode 44p and the voltage measurement electrode 44s in the third internal cavity 61, and further includes a pump reference electrode 42p and a voltage reference electrode 42s instead of the reference electrode 42 in FIG. 1. The pump reference electrode 42p and the voltage reference electrode 42s are both disposed inside the sensor element 401 so as to be in contact with a reference gas introduced into the reference-gas introduction portion 49. In this embodiment, as with the reference electrode 42, the pump reference electrode 42p and the voltage reference electrode 42s are covered by the reference-gas introduction layer 48. The pump reference electrode 42p constitutes part of the reference-gas adjustment pump cell 90, and the pump current Ip3 flows through the pump reference electrode 42p. The voltage reference electrode 42s constitutes part of each of the sensor cells 80 to 83. Thus, the voltage across the inner pump electrode 22 and the voltage reference electrode 42s gives the voltage V0, the voltage across the auxiliary pump electrode 51 and the voltage reference electrode 42s gives the voltage V1, the voltage across the pump measurement electrode 44p and the voltage reference electrode 42s gives the voltage V2, and the voltage across the outer pump electrode 23 and the voltage reference electrode 42s gives the voltage Vref. As with the pump measurement electrode 44p and the voltage measurement electrode 44s illustrated in FIG. 2, the pump reference electrode 42p and the voltage reference electrode 42s each have an approximately quadrangle shape in a top view. The voltage reference electrode 42s is located rearward of the pump reference electrode 42p. The voltage reference electrode 42s is shorter in length in the front-rear direction and smaller in area than the pump reference electrode 42p. Note that the areas of the pump reference electrode 42p and the voltage reference electrode 42s are each the area in a top view. The material for the pump reference electrode 42p and the voltage reference electrode 42s is the same as for the reference electrode 42 in the first embodiment. However, when the pump reference electrode 42p and the voltage reference electrode 42s contain a noble metal, the noble metal contained in the pump reference electrode 42p and the noble metal contained in the voltage reference electrode 42s may be different in at least one of type or content ratio.

Except this point, the gas sensor 400 is the same as the gas sensor 100 in the first embodiment. For example, the controller 96 controls the power supply circuit 92 to apply a voltage Vp3 repeatedly turned ON and OFF to the reference-gas adjustment pump cell 90, thereby causing the reference-gas adjustment pump cell 90 to pump oxygen into the periphery of the pump reference electrode 42p. The controller 96 obtains the voltages V0, V1, V2, Vref, and the pump currents Ip0 to Ip3 at the timing immediately before the subsequent turn-ON in an OFF-period of the voltage Vp3. The oxygen pumped into the periphery of the pump reference electrode 42p by the reference-gas adjustment pump cell 90 also reaches the periphery of the voltage reference electrode 42s through the reference-gas introduction layer 48. Thus, even when the pump reference electrode 42p and the voltage reference electrode 42s are separately provided in the reference-gas introduction portion 49, when the oxygen concentration in the periphery of the voltage reference electrode 42s decreases, the decreased oxygen can be supplemented by the reference-gas adjustment pump cell 90. Thus, when the measurement-object gas causes the oxygen concentration in the periphery of the voltage reference electrode 42s to decrease, it is possible to prevent change in the reference potential that is the electrical potential of the voltage reference electrode 42s, thus as in the first embodiment, reduction in the accuracy of detection of the voltages V0 to V2, Vref can be prevented by the reference-gas adjustment pump cell 90. Therefore, reduction in the accuracy of detection of the NOx concentration can also be prevented.

Of the correspondence relationships between the components in this embodiment and the components in the present invention, particularly, the correspondence relationships different from those in the first embodiment will now be clarified. The reference-gas introduction portion 49 in this embodiment corresponds to a reference-gas introduction portion of the present invention, the pump reference electrode 42p corresponds to a pump reference electrode, the reference-gas adjustment pump cell 90 corresponds to a reference-gas adjustment pump cell, and the voltage reference electrode 42s corresponds to a voltage reference electrode. In addition, the outer pump electrode 23 corresponds to a pumping-in source electrode, and the controller 96 corresponds to a reference-gas adjustment unit and a voltage acquisition unit.

In the gas sensor 400 in this embodiment described above in detail, the reference-gas adjustment pump cell 90 pumps oxygen into the periphery of the pump reference electrode 42p, thus reduction in the oxygen concentration of the reference gas in the reference-gas introduction portion 49 can be supplemented. In the V2 detection sensor cell 82, the voltage V2 based on the oxygen concentration difference between the reference gas and the third internal cavity 61 is generated, thus the oxygen concentration in the periphery of the voltage measurement electrode 44s can be detected with the voltage V2 of the V2 detection sensor cell 82. In the sensor element 401, the pump reference electrode 42p and the voltage reference electrode 42s are separately provided as the electrodes to be in contact with the reference gas in the reference-gas introduction portion 49. Thus, the same effect as the one achieved by separately providing the pump measurement electrode 44p and the voltage measurement electrode 44s in the above-described first embodiment is obtained. For example, unlike when one electrode 942 serves as the electrode of the reference-gas adjustment pump cell 990 as well as the electrode of the measurement-pump-control oxygen-partial-pressure detection sensor cell 982 as in the gas sensor 900 illustrated FIG. 17, the pump current Ip3 at the time of pumping-in of oxygen performed by the reference-gas adjustment pump cell 90 does not flow through the voltage reference electrode 42s of the sensor element 401. Thus, the voltage V2 of the measurement pump cell 41 does not include a voltage drop of the voltage reference electrode 42s due to the pump current Ip3. Consequently, in the sensor element 401, it is possible to prevent reduction in the accuracy of detection of the oxygen concentration in the third internal cavity 61 due to the pump current Ip3 at the time of pumping-in of oxygen, while oxygen is being pumped into the reference-gas introduction portion 49. Therefore, in the sensor element 401, the voltage V2 has a value which corresponds with higher accuracy to the oxygen concentration in the third internal cavity 61, thus the accuracy of detection the oxygen concentration in the third internal cavity 61 using the V2 detection sensor cell 82 is improved. In addition, even when a plurality of sensor elements 401 have a manufacturing variation in the voltage reference electrode 42s, the accuracy of detection of the oxygen concentration in the third internal cavity 61 with the voltage V2 is unlikely to have a variation.

Note that in the sensor element 401, as with the voltage V2, the voltages V0, V1, Vref also do not include a voltage drop of the voltage reference electrode 42s due to the pump current Ip3. Therefore, the voltages V0, V1, Vref have values which correspond with high accuracy to the oxygen concentration in the first internal cavity 20, the oxygen concentration in the second internal cavity 40, and the oxygen concentration in the measurement-object gas outside the sensor element 401, respectively. In addition, even when a plurality of sensor elements 401 have a manufacturing variation in the voltage reference electrode 42s, the accuracy of detection of the oxygen concentration in each of the first internal cavity 20, the second internal cavity 40, and the outside of the sensor element 401 is unlikely to have a variation.

The voltage V2 in the sensor element 401 is the voltage across the voltage measurement electrode 44s and the voltage reference electrode 42s, and in the gas sensor 400, no pump current flows through each of the voltage measurement electrode 44s and the voltage reference electrode 42s which are both-end electrodes for measurement of the voltage V2. Thus, in the sensor element 401, particularly, the voltage V2 has a value which corresponds with higher accuracy to the oxygen concentration than the voltages V0, V1, Vref. The voltage V2 of the sensor element 401 has a value which corresponds to the oxygen concentration in the third internal cavity 61 with an even higher accuracy than the voltage V2 of the sensor element 101.

### Fifth Embodiment

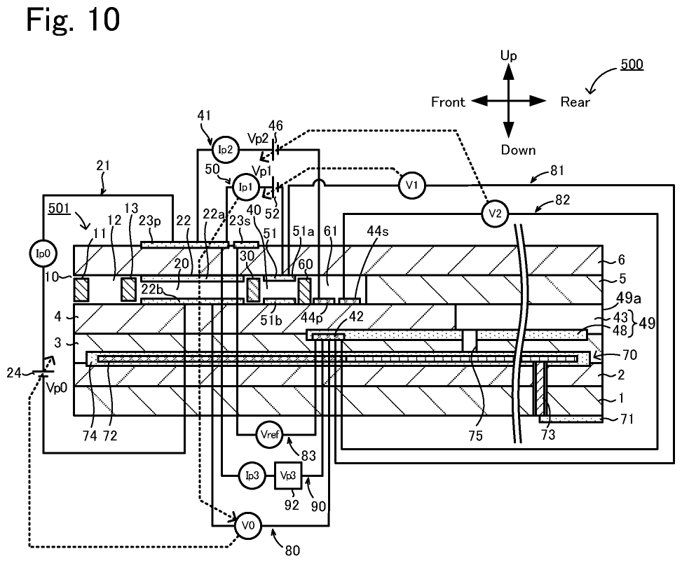

FIG. 10 is a schematic cross-sectional view schematically illustrating an example of the configuration of a gas sensor 500 in a fifth embodiment. As in the sensor element 101, a sensor element 501 of the gas sensor 500 includes the pump measurement electrode 44p and the voltage measurement electrode 44s in the third internal cavity 61, and further includes a pump outer electrode 23p and a voltage outer electrode 23s instead of the outer pump electrode 23 in FIG. 1. The pump outer electrode 23p and the voltage outer electrode 23s are both disposed outside the sensor element 501 so as to be in contact with the measurement-object gas outside the sensor element 501. In this embodiment, as with the outer pump electrode 23, the pump outer electrode 23p and the voltage outer electrode 23s are disposed on the upper surface of the sensor element 501. The pump outer electrode 23p constitutes part of each of the main pump cell 21, the auxiliary pump cell 50, the measurement pump cell 41, and the reference-gas adjustment pump cell 90, and pump currents Ip0, Ip1, Ip2, Ip3 flow through the pump outer electrode 23p. The voltage outer electrode 23s constitutes part of the Vref detection sensor cell 83. Thus, the voltage across the voltage outer electrode 23s and the reference electrode 42 is the voltage Vref. As with the pump measurement electrode 44p and the voltage measurement electrode 44s illustrated in FIG. 2, the pump outer electrode 23p and the voltage outer electrode 23s each have an approximately quadrangle shape in a top view. The voltage outer electrode 23s is located rearward of the pump outer electrode 23p. The voltage outer electrode 23s is shorter in length in the front-rear direction and smaller in area than the pump outer electrode 23p. The material for the pump outer electrode 23p and the voltage outer electrode 23s is the same as for the outer pump electrode 23 in the first embodiment. However, the noble metal contained in the pump outer electrode 23p and the noble metal contained in the voltage outer electrode 23s may be different in at least one of type or content ratio.

Except this point, the gas sensor 500 is the same as the gas sensor 100 in the first embodiment. For example, as in the first embodiment, the controller 96 feedback-controls the voltage Vp0 of the variable power supply 24 so that the voltage V0 reaches the target value V0*, thus the pump current Ip0 flows through the main pump cell 21. The controller 96 detects the oxygen concentration in the measurement-object gas outside the sensor element 501 based on the voltage Vref of the Vref detection sensor cell 83.

In the sensor element 501 of the gas sensor 500, as described above, the pump outer electrode 23p that constitutes part of each of the pump cells 21, 41, 50, 90, and the voltage outer electrode 23s that constitutes part of each of the Vref detection sensor cell 83 are both disposed outside the sensor element 501. In short, in the sensor element 501, the pump outer electrode 23p and the voltage outer electrode 23s are both disposed outside the sensor element 501. Thus, the same effect as the one achieved by separately providing the pump measurement electrode 44p and the voltage measurement electrode 44s in the above-described first embodiment is obtained. For example, unlike when one outer pump electrode 923 serves as the electrode of the measurement pump cell 941 as well as the electrode of the Vref detection sensor cell 983 as in the gas sensor 900 illustrated FIG. 17, the pump current Ip2 does not flow through the voltage outer electrode 23s. Similarly, the pump currents Ip0, Ip1, Ip3 do not flow through the voltage outer electrode 23s either. Thus, the voltage Vref of the Vref detection sensor cell 83 does not include a voltage drop of the voltage outer electrode 23s due to the pump currents Ip0 to Ip3. Consequently, the voltage Vref of the Vref detection sensor cell 83 has a value which corresponds with higher accuracy to the oxygen concentration in the measurement-object gas outside the sensor element 501, thus the accuracy of detection of the oxygen concentration in the measurement-object gas using the Vref detection sensor cell 83 is improved. In addition, even when a plurality of sensor elements 501 have a manufacturing variation in the voltage outer electrode 23s, the accuracy of detection of the oxygen concentration in the measurement-object gas outside the sensor element 501 with the voltage Vref is unlikely to have a variation.

As described above, the controller 96 controls the main pump cell 21 so that the voltage V0 reaches the target value V0*, in other words, the oxygen concentration in the first internal cavity 20 reaches a predetermined low concentration. In this situation, for example, when the oxygen concentration in the measurement-object gas is switched between a high state in which the oxygen concentration is higher than a predetermined low concentration and a low state, the controller 96 switches the direction of oxygen moved by the main pump cell 21 to the reverse direction. Thus, the direction of the pump current Ip0 which flows through the main pump cell 21 is switched to the reverse direction. For example, when the measurement-object gas is switched from a lean atmosphere to a rich atmosphere, the direction of the pump current Ip0 which flows through the main pump cell 21 is switched from the direction in which oxygen is pumped out from the first internal cavity 20 to the direction in which oxygen is pumped into the first internal cavity 20. The lean atmosphere indicates a state where the air-fuel ratio of the measurement-object gas is higher than a theoretical air-fuel ratio, and the rich atmosphere indicates a state where the air-fuel ratio of the measurement-object gas is lower than a theoretical air-fuel ratio. In a rich atmosphere, the measurement-object gas contains an unburnt fuel, and the right amount of oxygen required for burning the unburnt fuel corresponds to the oxygen concentration in the measurement-object gas in a rich atmosphere. Therefore, the oxygen concentration in the measurement-object gas in a rich atmosphere is expressed as a negative value. Thus, when the measurement-object gas is in a rich atmosphere, in order to change a negative oxygen concentration to a predetermined low concentration (a state where the oxygen concentration is higher than 0%) corresponding to the target value V0*, the controller 96 controls the main pump cell 21 to pump oxygen into the first internal cavity 20. Thus, when one electrode serves as the pump outer electrode 23p as well as the voltage outer electrode 23s, the change in the voltage Vref also becomes slow due to the time required for current change when the direction of the pump current Ip0 flowing through the main pump cell 21 is switched to the reverse direction. In contrast, in this embodiment, the pump outer electrode 23p and the voltage outer electrode 23s are separately provided, thus the voltage Vref is not affected by the time required for change in the pump current Ip0, and therefore, the change in the voltage Vref does not become slow. In other words, when the oxygen concentration in the measurement-object gas is switched between a high state in which the oxygen concentration is higher than a predetermined low concentration and a low state, the responsiveness of the voltage Vref is not likely to reduce.

In addition, when one electrode serves as the pump outer electrode 23p as well as the voltage outer electrode 23s, the electrode deteriorates with use, thus the aforementioned time required for current change when the direction of the pump current Ip0 is switched to the reverse direction may be further increased. This is probably because capacity components of the electrode change due to deterioration of the electrode. Thus, for example, in the gas sensor 900, the responsiveness of the voltage Vref may reduce with use (hereinafter referred to as “deterioration of responsiveness”). In contrast, in this embodiment, the voltage outer electrode 23s is unlikely to deteriorate because the pump currents Ip0 to Ip3 are not passed through the voltage outer electrode 23s. Even if the voltage outer electrode 23s deteriorates, the pump current Ip0 is not passed through the voltage outer electrode 23s, thus the voltage outer electrode 23s is not affected by switching of the direction of the pump current Ip0 to the reverse direction. Consequently, even when the sensor element 501 is used for a long time, the responsiveness of the voltage Vref is unlikely to deteriorate.

The responsiveness of the voltage Vref and the manner of deterioration of the responsiveness have been studied in the following way. First, Example 2 is implemented by producing the sensor element 501 and the gas sensor 500 in this embodiment illustrated in FIG. 10. In addition, Example 3 is implemented by producing a gas sensor which is the same as Example 2 except that the pump outer electrode 23p and the voltage outer electrode 23s are not included but the outer pump electrode 923 of FIG. 17 is included. In Example 3, the outer pump electrode 923 constitutes part of each of the main pump cell 21, the auxiliary pump cell 50, the measurement pump cell 41, the reference-gas adjustment pump cell 90, and the Vref detection sensor cell 83. The same material is used for the pump outer electrode 23p, and the voltage outer electrode 23s in Example 2, and the outer pump electrode 923 in Example 3.

For Examples 2, 3, the responsiveness of the voltage Vref was studied. First, the gas sensor in Example 2 was mounted on a pipe. The heater 72 was energized to attain a temperature of 800° C. to heat the sensor element 501. A state is achieved in which the aforementioned pump cells 21, 41, 50 are controlled by the controller 96, and the voltages V0, V1, V2, Vref are obtained from the aforementioned sensor cells 80 to 83. A state is achieved in which the reference-gas adjustment pump cell 90 is not controlled by the controller 96. In this state, as a measurement-object gas, a gas simulating an exhaust gas in a lean state is passed through a pipe, and subsequently, a gas simulating an exhaust gas in a rich state is passed through the pipe, thus switching of the measurement-object gas from a lean state to a rich state was simulated. The voltage Vref then was continuously measured, and the manner of temporal change in the voltage Vref was studied. Similarly, also for Example 3, the manner of temporal change in the voltage Vref was studied.

Specifically, when the gas to be passed through the pipe is switched from a lean state to a rich state, the voltage Vref rose in each of Examples 2, 3. The value of the voltage Vref immediately before rise thereof is assumed to be 0%, the value of the voltage Vref after being stabilized after the rise is assumed to be 100%, and the response time [msec] of the voltage Vref is defined by the time required for the voltage Vref to change from 10% to 90%. A shorter response time indicates a higher responsiveness of the voltage Vref. The response time in Example 2 was 380 msec, and the response time in Example 3 was 400 msec. From this result, it was verified that the responsiveness of rising of the voltage Vref is higher in Example 2 in which the pump outer electrode 23p and the voltage outer electrode 23s are both provided than in Example 3 in which the outer pump electrode 923 is disposed instead of these electrodes. The responsiveness of falling of the voltage Vref at the time of switching the gas to be passed through the pipe from a rich state to a lean state was studied in the same manner, and the responsiveness was higher in Example 2 than in Example 3.

Next, in a state where the gas sensor 500 in Example 2 was placed in the atmosphere, a continuous test in atmosphere was conducted in the same manner as described above, that is, the sensor element 501 was driven by the controller 96 to operate until 500 hours elapsed. For the gas sensor in Example 3, a continuous test in atmosphere was also conducted in the same manner. The atmosphere is higher in oxygen concentration than the exhaust gas, and the noble metal in the electrode is likely to be oxidized and deteriorated, thus the continuous test in atmosphere is an accelerated deterioration test for electrode. For Examples 2, 3 after the continuous test in atmosphere was conducted, the response time [msec] of the voltage Vref was measured by the aforementioned method.

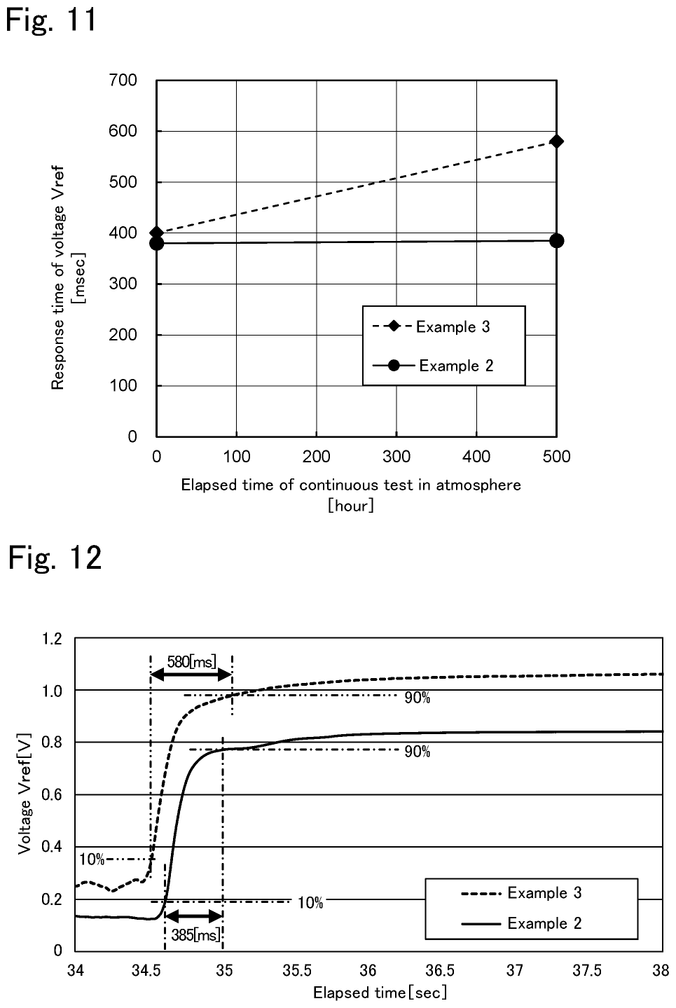

FIG. 11 shows graphs illustrating the change in response time of the voltage Vref before and after the continuous test in atmosphere in Examples 2, 3. As illustrated in FIG. 11, in Example 3, the response time (580 msec) after the continuous test in atmosphere (elapsed time is 500 hours) is longer than the response time (400 msec) before the continuous test in atmosphere (elapsed time is 0 hour), that is, the responsiveness has deteriorated. In contrast, in Example 2, the response time changed from 380 msec to 385 msec only before and after the continuous test in atmosphere, thus change in the response time was little. From this result, it was verified that deterioration of the response time of the voltage Vref with use of the gas sensor is further reduced in Example 2 in which the pump outer electrode 23p and the voltage outer electrode 23s are both provided than in Example 3 in which the outer pump electrode 923 is disposed instead of these electrodes. FIG. 12 shows graphs illustrating the manner of temporal change in the voltage Vref in Examples 2, 3 after the continuous test in atmosphere. In FIG. 12, the voltages Vref corresponding to 10% and 90% are shown for each of Examples 2, 3, where the value of the voltage Vref immediately before rise thereof is assumed to be 0%, and the value of the voltage Vref after being stabilized after the rise is assumed to be 100%. In addition, in FIG. 12, the value of the aforementioned response time was shown for each of Examples 2, 3, where the response time was measured as the time required for the voltage Vref to change from 10% to 90%.

Note that the sensor element in Example 3 has substantially the same configuration as that of the sensor element 101. Not only in Example 2 but also in Example 3, the pump measurement electrode 44p and the voltage measurement electrode 44s are provided, thus, the same effect as that of the gas sensor 100 of the above-described first embodiment is achieved. Therefore, Example 3 is not a comparative example, and corresponds to an example of the present invention.

When the controller 96 detects the oxygen concentration in the measurement-object gas outside the sensor element 501 based on the voltage Vref of the Vref detection sensor cell 83, as a kind of detection of the oxygen concentration, whether the measurement-object gas outside the sensor element 501 is in a rich state or a lean state may be determined based on the voltage Vref. For example, a predetermined threshold to determine whether the voltage Vref is in a rising state or a falling state is pre-stored in the storage unit 98, and the controller 96 may binarize an obtained voltage Vref based on the threshold to determine whether the measurement-object gas is in a rich state or a lean state. In this manner, the gas sensor 500 functions not only as an NOx sensor but also as a lambda sensor (air-fuel ratio sensor). Note that in the gas sensor 100 in the first embodiment also, the controller 96 may determine whether the measurement-object gas is in a rich state or a lean state in the same manner as described above.

Of the correspondence relationships between the components in this embodiment and the components in the present invention, particularly, the correspondence relationships different from those in the first embodiment will now be clarified. The voltage outer electrode 23s in this embodiment corresponds to a voltage outer electrode of the present invention, the Vref detection sensor cell 83 corresponds to an outer sensor cell, and each of the main pump cell 21, the auxiliary pump cell 50, and the measurement pump cell 41 corresponds to a flow portion pump cell. In addition, the reference electrode 42 corresponds to a reference electrode, the main pump cell 21 corresponds to an adjustment chamber pump cell, and the controller 96 corresponds to an adjustment chamber pump cell controller and an oxygen concentration detector.

In the gas sensor 500 in this embodiment described above in detail, the pump outer electrode 23p and the voltage outer electrode 23s are separately provided outside the sensor element 501. Accordingly, the pump currents Ip0 to Ip3 do not flow through the voltage outer electrode 23s, thus the voltage Vref of the Vref detection sensor cell 83 does not include a voltage drop of the voltage outer electrode 23s due to the pump currents Ip0 to Ip3. Consequently, the voltage Vref has a value which corresponds with higher accuracy to the oxygen concentration in the measurement-object gas outside the sensor element 501, thus the accuracy of detection of the oxygen concentration in the measurement-object gas using the Vref detection sensor cell 83 is improved.

The controller 96 causes the main pump cell 21 to pump out oxygen from the first internal cavity 20 or pump oxygen into the first internal cavity 20 by controlling the main pump cell 21 so that the oxygen concentration reaches a predetermined low concentration. In this case, the direction of the pump current Ip0 which flows through the main pump cell 21 may be switched to the reverse direction. However, since the pump outer electrode 23p and the voltage outer electrode 23s are separately provided in the sensor element 501, the voltage Vref is not affected by the time required for change in the pump current Ip0. Consequently, when the oxygen concentration in the measurement-object gas is switched between a high state in which the oxygen concentration is higher than a predetermined low concentration and a low state, the responsiveness of the voltage Vref is not likely to reduce.

The present invention is not limited whatsoever to the above embodiments, and various embodiments are possible so long as they belong within the technical scope of the present invention.

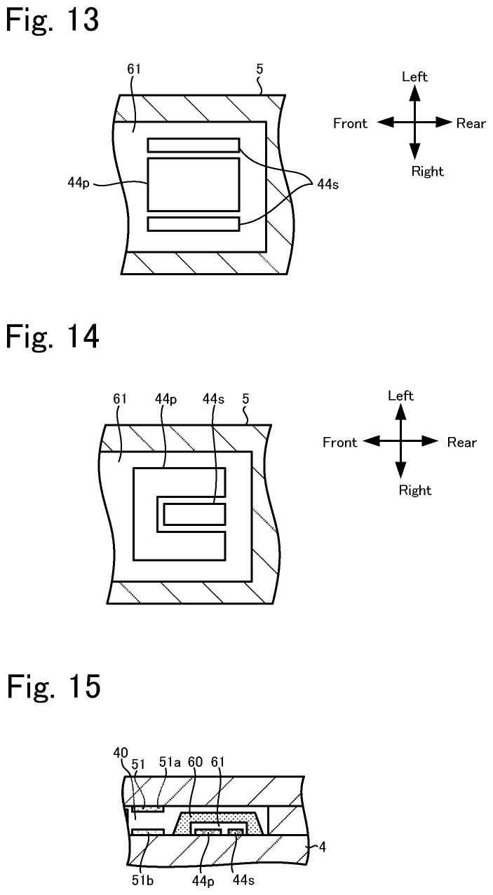

For example, in the first to fifth embodiments described above, the pump measurement electrode 44p and the voltage measurement electrode 44s are disposed side by side in the front-rear direction, however, may be disposed side by side in the left-right direction. As illustrated in FIG. 13, the voltage measurement electrode 44s may be disposed on both the right and left of the pump measurement electrode 44p. The two voltage measurement electrodes 44s illustrated in FIG. 13 are electrically connected by a lead wire which is not illustrated, and function as one voltage measurement electrode. As illustrated in FIG. 14, the pump measurement electrode 44p may have a recessed portion, and the voltage measurement electrode 44s may be disposed in the recessed portion. In this manner, the voltage measurement electrode 44s is surrounded by the pump measurement electrode 44p in three directions among the front and left-right directions, thus the oxygen concentration in the periphery of the pump measurement electrode 44p can be detected with high accuracy using the voltage V2. The pump measurement electrode 44p and the voltage measurement electrode 44s may be disposed side by side in the up-down direction. For example, the voltage measurement electrode 44s may be disposed on the lower surface of the second solid electrolyte layer 6 instead of the upper surface of the first solid electrolyte layer 4 as in FIG. 1. However, as described above, the pump measurement electrode 44p and the voltage measurement electrode 44s are preferably disposed as close as possible, thus as illustrated in FIGS. 1, 2, 13, 14, the pump measurement electrode 44p and the voltage measurement electrode 44s are preferably disposed on the same surface of the same solid electrolyte layer.

The aforementioned various embodiments of the pump measurement electrode 44p and the voltage measurement electrode 44s including FIGS. 2, 13, 14 may be applied to the embodiment of the pump auxiliary electrode 51p and the voltage auxiliary electrode 51s, the embodiment of the pump main electrode 22p and the voltage main electrode 22s, the embodiment of the pump reference electrode 42p and the voltage reference electrode 42s, and the embodiment of the pump outer electrode 23p and the voltage outer electrode 23s. However, the pump outer electrode 23p and the voltage outer electrode 23s do not need to be disposed close to each other. It is preferable that the pump outer electrode 23p and the voltage outer electrode 23s be disposed with a certain gap therebetween so that the voltage Vref does not change due to the effect of the oxygen pumped out into the periphery of the pump outer electrode 23p.

In the above-described first embodiment, it has been explained that the voltage measurement electrode 44s is preferably reduced in area to lower the thermal electromotive force. Similarly, the voltage auxiliary electrode 51s, the voltage main electrode 22s, the voltage reference electrode 42s, and the voltage outer electrode 23s are preferably reduced in area to lower the thermal electromotive force.

In the above-described second embodiment, the pump auxiliary electrode 51p and the voltage auxiliary electrode 51s each have a structure in a tunnel form, but are not limited thereto. For example, the voltage auxiliary electrode 51s may not have a tunnel form, and may be disposed only on the upper surface of the first solid electrolyte layer 4, or disposed only on the lower surface of the second solid electrolyte layer 6. The same applies to the pump main electrode 22p and the voltage main electrode 22s in the third embodiment.

In the above-described first embodiment, the fourth diffusion control section 60 is formed as a slit-shaped gap, but is not limited thereto. The fourth diffusion control section 60 may be formed as a porous body (e.g., a ceramic porous body such as alumina (Al₂O₃)). For example, as illustrated in FIG. 15, the third internal cavity 61 may be the space surrounded by the first solid electrolyte layer 4, and the fourth diffusion control section 60 formed as a porous body, and the pump measurement electrode 44p and the voltage measurement electrode 44s may be disposed in the third internal cavity 61. The third internal cavity 61 as the space surrounded by such a porous body can be formed using a paste composed of a disappearing material (e.g., theobromine) that disappears in a calcination process.

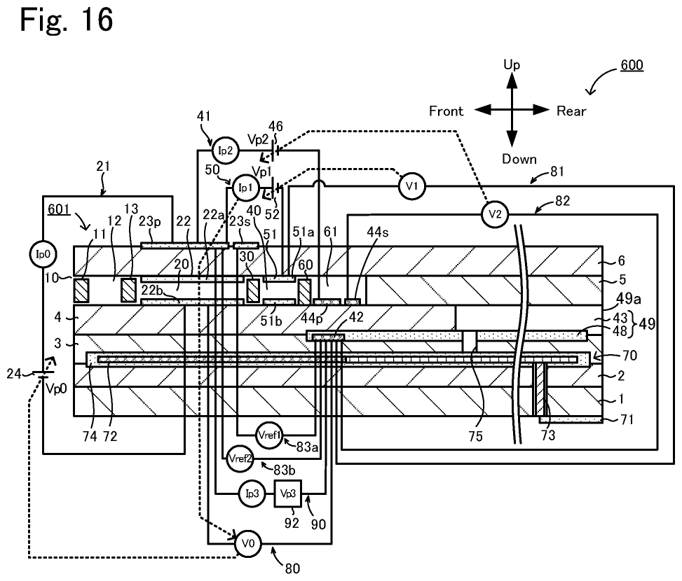

In the above-described fifth embodiment, the controller 96 may obtain not only the voltage Vref across the voltage outer electrode 23s and the reference electrode 42, but also the voltage across the pump outer electrode 23p and the reference electrode 42. FIG. 16 is a schematic cross-sectional view of a gas sensor 600 according to a modification. A sensor element 601 of the gas sensor 600 includes a Vref1 detection sensor cell 83a and a Vref2 detection sensor cell 83b. The Vref1 detection sensor cell 83a is the same sensor cell as the Vref detection sensor cell 83 of the sensor element 501. In the Vref1 detection sensor cell 83a, a voltage Vref1 is generated between the voltage outer electrode 23s and the reference electrode 42. The Vref2 detection sensor cell 83b is an electrochemical sensor cell including: the second solid electrolyte layer 6, the spacer layer 5, the first solid electrolyte layer 4, the third substrate layer 3, the pump outer electrode 23p, and the reference electrode 42. In the Vref2 detection sensor cell 83b, a voltage Vref2 is generated between the pump outer electrode 23p and the reference electrode 42. The gas sensor 600 can determine whether the pump outer electrode 23p is deteriorated based on the difference between the voltage Vref1 and the voltage Vref2. For example, the controller 96 obtains a current Ip4 (e.g., the total value of the pump currents Ip0 to Ip3) which flows through the pump outer electrode 23p, the voltage Vref1 and the voltage Vref2 at a predetermined deterioration determination timing, and calculates the difference Da between the voltage Vref1 and the voltage Vref2 obtained. Next, the controller 96 calculates a reference value for the difference between the voltage Vref1 and the voltage Vref2 based on the obtained current Ip4. The reference value is a value corresponding to the difference between the voltage Vref1 and the voltage Vref2 in a state where the pump outer electrode 23p is not deteriorated. The difference between the voltage Vref1 and the voltage Vref2 includes a voltage drop in the pump outer electrode 23p due to the current which flows through the pump outer electrode 23p, and the controller 96 calculates a reference value based on the obtained pump current Ip4. For example, a relational expression (e.g., the expression of a linear function) and a map representing a correspondence relationship between the current Ip4 and the reference value are pre-stored in the storage unit 98, and the controller 96 calculates a reference value using the obtained current Ip4 and the correspondence relationship. Note that when the rate of the current Ip0 to the current Ip4 (the total value of the currents Ip0 to Ip3) is high, a reference value may be calculated based on the current Ip0 rather than the current Ip4. It is determined whether the pump outer electrode 23p is deteriorated based on whether the difference Da deviates from the reference value (e.g., whether the difference between the difference Da and the reference value exceeds a predetermined threshold). The pump currents Ip0 to Ip3 flow through the pump outer electrode 23p with use of the sensor element 601, thus the pump outer electrode 23p deteriorates. Thus, even when the current which flows through the pump outer electrode 23p is in the same state as before the deterioration, the voltage drop in the pump outer electrode 23p due to the current flow is increased than before the deterioration. Thus, the difference Da between the voltage Vref1 and the voltage Vref2 tends to increase as the pump outer electrode 23p deteriorates. Therefore, the controller 96 can determine whether the pump outer electrode 23p is deteriorated by comparing the difference Da with the aforementioned reference value. When the pump outer electrode 23p deteriorates, the accuracy of measurement of the NOx concentration may be reduced by a change in the values of the pump currents Ip0 to Ip3 which are caused to flow by respective voltages Vp0 to Vp3. When the controller 96 is able to determine deterioration of the pump outer electrode 23p, for example, the controller 96 can prevent the accuracy of measurement of the NOx concentration from remaining at a low level through handling such as transmission of error information to an engine ECU. Note that the controller 96 can determine not only whether the pump outer electrode 23p is deteriorated, but also the degree of deterioration of the pump outer electrode 23p based on the magnitude of the difference Da, or based on the degree of deviation (e.g., the magnitude of the difference between the difference Da and the reference value) between the difference Da and the reference value. In addition, the controller 96 may change control of the sensor element 601 so that effect of deterioration is canceled according to presence or absence of deterioration or the degree of deterioration of the pump outer electrode 23p. For example, the controller 96 may change at least one of the aforementioned target values V0*, V1*, V2*, or Ip1* based on the difference Da or based on the difference between the difference Da and the reference value. Alternatively, the controller 96 may change the amount of oxygen pumped into the periphery of reference electrode 42 by changing the voltage Vp3 to change the pump current Ip3 based on the difference Da or based on the difference between the difference Da and the reference value.

In the above-described first embodiment, the sensor element 101 may not include the reference gas adjustment pump cell 90, and the controller 96 may not include the power supply circuit 92, so that pumping of oxygen into the periphery of the reference electrode 42 by the reference-gas adjustment pump cell 90 may not be provided. The same applies to the second, third, and fifth embodiments. Note that when the reference-gas adjustment pump cell 90 pumps oxygen into the reference-gas introduction portion 49, not only the pump currents Ip0 to Ip2 but also the pump current Ip3 flow through the outer pump electrode 23, thus the current flowing through the outer pump electrode 23 is increased, and the outer pump electrode 23 is likely to deteriorate. Thus, when the reference-gas adjustment pump cell 90 pumps in oxygen, high significance is given to prevention of deterioration of the responsiveness of the voltage Vref by separately providing the pump outer electrode 23p and the voltage outer electrode 23s as in the fifth embodiment.

In the above-described first to fourth embodiments, the reference-gas adjustment pump cell 90 includes the outer pump electrode 23 disposed outside the element body as a pumping-in source electrode which serves as a source to pump oxygen into the reference-gas introduction portion 49. Similarly, in the above-described fifth embodiment, as the pumping-in source electrode, the pump outer electrode 23p disposed outside the element body is provided. However, without being limited to this, the pumping-in source electrode may be disposed inside or outside the element body so as to be in contact with the measurement-object gas. For example, the inner pump electrode 22 in FIG. 1 may be used as a pumping-in source electrode, and the reference-gas adjustment pump cell 90 may pump oxygen into the reference-gas introduction portion 49 from the periphery of the inner pump electrode 22. The reference-gas adjustment pump cell 90 may pump out oxygen from the periphery (the periphery of the pump reference electrode 42p in the fourth embodiment) of the reference electrode 42.

In the above-described first embodiment, the element body of the sensor element 101 is a layered body having a plurality of solid electrolyte layers (layers 1 to 6), but is not limited thereto. The element body of the sensor element 101 may include at least one oxygen-ion-conductive solid electrolyte layer, and may be internally provided with a measurement-object gas flow portion. For example, in FIG. 1, the layers 1 to 5 other than the second solid electrolyte layer 6 may be structural layers (e.g., layers composed of alumina) composed of a material other than that of solid electrolyte layers. In this case, the electrodes possessed by the sensor element 101 may be disposed in the second solid electrolyte layer 6. For example, the pump measurement electrode 44p and the voltage measurement electrode 44s in FIG. 1 may be disposed on the lower surface of the second solid electrolyte layer 6. Also, the reference-gas introduction space 43 may be provided in the spacer layer 5 instead of the first solid electrolyte layer 4, the reference-gas introduction layer 48 may be provided between the second solid electrolyte layer 6 and the spacer layer 5 instead of between the first solid electrolyte layer 4 and the third substrate layer 3, and the reference electrode 42 may be provided rearward of the third internal cavity 61 and on the lower surface of the second solid electrolyte layer 6. The same applies to the second to fifth embodiments.

In the above-described first to fifth embodiments, the controller 96 sets (feedback-controls) the target value V0* of the voltage V0 based on the pump current Ip1 so that the pump current Ip1 reaches the target value Ip1*, and the controller 96 feedback-controls the voltage Vp0 so that the voltage V0 reaches the target value V0*, but may perform another control. For example, the controller 96 may feedback-control the voltage Vp0 based on the pump current Ip1 so that the pump current Ip1 reaches the target value Ip1*. In other words, the controller 96 may directly control the voltage Vp0 (eventually control the pump current Ip0) based on the pump current Ip1 without obtaining the voltage V0 from the V0 detection sensor cell 80 and setting the target value V0*. Also, in this situation, the controller 96 feedback-controls the voltage Vp1 so that the voltage V1 reaches the target value V1*, thus the controller 96 controls the oxygen concentration in the first internal cavity 20 upstream of the second internal cavity 40 at a predetermined low concentration using the main pump cell 21 so that the pump current Ip1 reaches the target value Ip1* and the oxygen concentration in the second internal cavity 40 reaches a predetermined low concentration (an oxygen concentration corresponding to the voltage V1). Therefore, even when control according to such a modification is performed, as in the description of the fifth embodiment, when the oxygen concentration in the measurement-object gas is switched between a high state in which the oxygen concentration is higher than a predetermined low concentration and a low state, the direction of the pump current Ip0 is switched to the reverse direction. Thus, even when control according to such a modification is performed, the effect of preventing reduced responsiveness of the voltage Vref is obtained as in the fifth embodiment described above by separately providing the pump outer electrode 23p and the voltage outer electrode 23s as in the fifth embodiment.

In the above-described first embodiment, the oxygen concentration adjustment chamber has the first internal cavity 20 and the second internal cavity 40, however, without being limited to this, for example, the oxygen concentration adjustment chamber may include a still another internal cavity, or one of the first internal cavity 20 and the second internal cavity 40 may be omitted. Similarly, in the above-described first embodiment, the adjustment pump cell has the main pump cell 21 and the auxiliary pump cell 50, however, without being limited to this, for example, the adjustment pump cell may include a still another pump cell, and one of the main pump cell 21 and the auxiliary pump cell 50 may be omitted. For example, when the oxygen concentration in the measurement-object gas can be sufficiently reduced to a low oxygen concentration only by the main pump cell 21, the auxiliary pump cell 50 may be omitted. When the auxiliary pump cell 50 is omitted, the controller 96 may omit the aforementioned setting of the target value V0* based on the pump current Ip1. Specifically, a predetermined target value V0* is pre-stored in the storage unit 98, and the controller 96 may control the main pump cell 21 by feedback-controlling the voltage Vp0 of the variable power supply 24 so that the voltage V0 reaches the target value V0*. The same applies to the second to fifth embodiments. Particularly, in the embodiment in which the pump main electrode 22p and the voltage main electrode 22s are provided as in the third embodiment illustrated in FIG. 8, the accuracy of detection of the oxygen concentration in the first internal cavity 20 using the V0 detection sensor cell 80 is improved as described above, thus a configuration is easily used in which the second internal cavity 40 and the auxiliary pump cell 50 are omitted. The manufacturing cost of the sensor element 101 can be reduced by omitting the second internal cavity 40 and the auxiliary pump cell 50 (particularly, the auxiliary pump electrode 51, the pump auxiliary electrode 51p, the voltage auxiliary electrode 51s).

In the above-described first embodiment, the gas sensor 100 detects the NOx concentration as a specific gas concentration, however, without being limited to this, another oxide concentration may be used as a specific gas concentration. In the case where the specific gas is an oxide, when the specific gas itself is reduced in the third internal cavity 61, oxygen is produced as in the above-described first embodiment, thus the controller 96 can detect a specific gas concentration based on the detection value according to the oxygen. Alternatively, the specific gas may be a non-oxide such as ammonia. In the case where the specific gas is a non-oxide, when the specific gas is converted to an oxide (e.g., ammonia is oxidized and converted to NO), for example, in the first internal cavity 20, and the converted oxide is reduced in the third internal cavity 61, oxygen is produced, thus the controller 96 can obtain a detection value according to the oxygen and detect a specific gas concentration. In this manner, regardless of whether the specific gas is an oxide or a non-oxide, the gas sensor 100 can detect a specific gas concentration based on the oxygen produced from the specific gas in the third internal cavity 61. The same applies to the second to fifth embodiments.

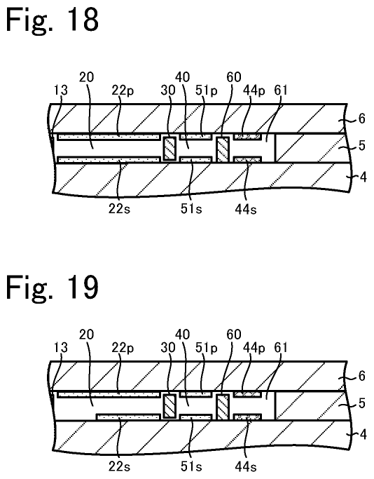

As described above, the pump measurement electrode 44p and the voltage measurement electrode 44s may be disposed side by side in the up-down direction, and in that situation, the voltage measurement electrode 44s and the pump measurement electrode 44p may be disposed so that the solid electrolyte layer in which the voltage measurement electrode 44s is disposed is located closer to the heater 72 than the solid electrolyte layer in which the pump measurement electrode 44p is disposed. For example, as illustrated in FIG. 18, the voltage measurement electrode 44s may be disposed on the upper surface of the first solid electrolyte layer 4, and the pump measurement electrode 44p may be disposed on the lower surface of the second solid electrolyte layer 6 which is further from the heater 72 than the first solid electrolyte layer 4. Temperature rises at the start of driving of the sensor element 101 more quickly by locating the first solid electrolyte layer 4 in which the voltage measurement electrode 44s is disposed closer to the heater 72 than the second solid electrolyte layer 6 in which the pump measurement electrode 44p is disposed. Therefore, the first solid electrolyte layer 4 is activated earlier than the second solid electrolyte layer 6 at the start of driving of the sensor element 101, thus detection of the voltage V2 using the voltage measurement electrode 44s can be started early. In short, light-off of the V2 detection sensor cell 82 is made quicker. In addition, the pump measurement electrode 44p and the second solid electrolyte layer 6 are located further from the heater 72 than the first solid electrolyte layer 4, thus the temperature of the pump measurement electrode 44p during use of the sensor element 101 is maintained at a temperature lower than the temperature of the voltage measurement electrode 44s. Thus, deterioration (reduction in catalytic activity) of the pump measurement electrode 44p is prevented, and deterioration of the accuracy of detection of the NOx concentration is prevented. Note that the temperature of the voltage measurement electrode 44s during use of the sensor element 101 is maintained at a temperature higher than the temperature of the pump measurement electrode 44p, and as described above, even if the voltage measurement electrode 44s deteriorates, the pump current Ip2 is not passed therethrough, thus a voltage drop does not occur. Therefore, the accuracy of detection of NOx concentration is unlikely to be affected. The same applies to the disposition of the pump auxiliary electrode 51p and the voltage auxiliary electrode 51s as well as the disposition of the pump main electrode 22p and the voltage main electrode 22s. FIG. 18 illustrates an example when these electrodes are disposed side by side in the up-down direction. Note that in FIG. 18, the pump auxiliary electrode 51p and the voltage auxiliary electrode 51s are disposed in the up-down direction, thus unlike FIG. 7, the pump auxiliary electrode 51p and the voltage auxiliary electrode 51s do not have a structure in a tunnel form. Specifically, each of the pump auxiliary electrode 51p and the voltage auxiliary electrode 51s in FIG. 18 does not include a lateral electrode portion. Thus, when the sensor element 101 is produced, the pump auxiliary electrode 51p and the voltage auxiliary electrode 51s are easily manufactured, and the effect of reducing the manufacturing cost of the sensor element 101 is also obtained. The same applies to the pump main electrode 22p and the voltage main electrode 22s in FIG. 18.

When the pump main electrode 22p and the voltage main electrode 22s are disposed in the up-down direction as in FIG. 18, the voltage main electrode 22s may be disposed so that the upstream end of the voltage main electrode 22s is located downstream of the upstream end of the pump main electrode 22p. For example, as illustrated in FIG. 19, the upstream end (in this case, the front end) of the voltage main electrode 22s may be located downstream (in this case, rearward) of the upstream end (in this case, the front end) of the pump main electrode 22p by reducing the length of the voltage main electrode 22s in the front-rear direction. In this manner, the measurement-object gas after pumping-out of oxygen into the periphery of the pump main electrode 22p by the pump current Ip0 reaches the voltage main electrode 22s. In other words, the voltage main electrode 22s is disposed away from an area where the oxygen concentration is likely to increase. Since the voltage main electrode 22s is disposed away from an area where the oxygen concentration is likely to increase, when the voltage main electrode 22s contains Au, evaporation of Au from the voltage main electrode 22s with use of the gas sensor 100 can be prevented. When Au is evaporated from the voltage main electrode 22s, the Au may adhere to the pump measurement electrode 44p and/or the voltage measurement electrode 44s to reduce the catalytic activity of these electrodes, thus NOx cannot be sufficiently reduced in the periphery of these electrodes. As a result, the accuracy of detection of the NOx concentration of the gas sensor 100 may decrease. Such decrease in the accuracy of detection of the NOx concentration can be prevented by reducing the evaporation of Au from the voltage main electrode 22s. Note that as the noble metal in an electrode is oxidized, Au is more likely to be evaporated from the electrode. For example, in an electrode containing Pt and Au, with a higher oxygen concentration, Pt is more likely to be oxidized to produce PtO₂. PtO₂ has a higher saturated vapor pressure than that of Pt, thus is more likely to be evaporated than Pt. When Pt is evaporated in the form of PtO₂, the remaining Au is also likely to be evaporated. This is because single-component Au has a higher saturated vapor pressure than that of Pt—Au alloy. The noble metal in an electrode is more likely to be oxidized at a higher oxygen concentration in the periphery of the electrode and more current flow through the electrode. In FIG. 19, as described above, the voltage main electrode 22s is disposed away from an area where the oxygen concentration is likely to increase, thus evaporation of Au from the voltage main electrode 22s can be prevented. Although the pump main electrode 22p is not disposed away from an area where the oxygen concentration is likely to increase, the pump main electrode 22p is located further from the heater 72 than the voltage main electrode 22s, thus the temperature of the pump main electrode 22p during use of the sensor element 101 is maintained at a temperature lower than the temperature of the voltage main electrode 22s. Therefore, oxidation of the noble metal in the pump main electrode 22p is prevented, thus evaporation of Au from the pump main electrode 22p is also prevented. Note that the voltage main electrode 22s may be disposed so that the upstream end of the voltage main electrode 22s may be located downstream of the downstream end of the pump main electrode 22p. In other words, the entire voltage main electrode 22s may be located downstream of the pump main electrode 22p.

The above-described first to fifth embodiments and various modifications may be combined as appropriate. For example, in the fourth, fifth embodiments, the pump measurement electrode 44p and the voltage measurement electrode 44s are separately provided as in the first embodiment, however, in addition to or substitution of this, an aspect of the second embodiment, in which the pump auxiliary electrode 51p and the voltage auxiliary electrode 51s are separately provided may be used or an aspect of the third embodiment, in which the pump main electrode 22p and the voltage main electrode 22s are separately provided may be used. Note that of the voltages V0, V1, V2, the voltage V2 affects the most on the accuracy of detection of a specific gas concentration, thus particularly, the first embodiment is preferable between the first to third embodiments. In other words, it is preferable that the pump measurement electrode 44p and the voltage measurement electrode 44s be separately provided at least in the sensor element. In addition, all the aspects of the first to fifth embodiments may be combined. Specifically, in the sensor element 101 of FIG. 1, each of the inner pump electrode 22, the outer pump electrode 23, the auxiliary pump electrode 51, and the reference electrode 42 may be divided into a pump electrode and a voltage electrode as described in the second to fifth embodiments.

What is claimed is:

1. A sensor element for detecting a specific gas concentration in a measurement-object gas, the sensor element comprising:
an element body including an oxygen-ion-conductive solid electrolyte layer and internally provided with a measurement-object gas flow portion that introduces a measurement-object gas and causes the measurement-object gas to flow therethrough;
a flow portion pump cell having a pump inner electrode disposed in an internal cavity of the measurement-object gas flow portion, the flow portion pump cell being configured to pump out oxygen from the internal cavity or pump oxygen into the internal cavity; and
a flow portion sensor cell having a voltage inner electrode disposed in the internal cavity, the flow portion sensor cell being configured to generate a voltage based on an oxygen concentration in the internal cavity.

2. The sensor element according to claim 1, further comprising:
an adjustment chamber pump cell that adjusts an oxygen concentration in an oxygen concentration adjustment chamber of the measurement-object gas flow portion,
wherein the internal cavity is a measurement chamber provided downstream of the oxygen concentration adjustment chamber in the measurement-object gas flow portion,
the pump inner electrode is a pump measurement electrode disposed in the measurement chamber,
the voltage inner electrode is a voltage measurement electrode disposed in the measurement chamber,
the flow portion pump cell is a measurement pump cell that pumps out oxygen produced from the specific gas in the measurement chamber, and
the flow portion sensor cell is a measurement sensor cell that generates a voltage based on an oxygen concentration in the measurement chamber.

3. The sensor element according to claim 1, further comprising:
a measurement pump cell that pumps out oxygen from the measurement chamber of the measurement-object gas flow portion, the oxygen being produced from the specific gas in the measurement chamber,
wherein the internal cavity is an oxygen concentration adjustment chamber provided upstream of the measurement chamber in the measurement-object gas flow portion,
the pump inner electrode is a pump adjustment electrode disposed in the oxygen concentration adjustment chamber,
the voltage inner electrode is a voltage adjustment electrode disposed in the oxygen concentration adjustment chamber,
the flow portion pump cell is an adjustment chamber pump cell that adjusts an oxygen concentration in the oxygen concentration adjustment chamber, and
the flow portion sensor cell is an adjustment chamber sensor cell that generates a voltage based on the oxygen concentration in the oxygen concentration adjustment chamber.

4. The sensor element according to claim 1, further comprising:
a reference-gas introduction portion disposed inside the element body, the reference-gas introduction portion being configured to introduce a reference gas serving as a reference for detecting a specific gas concentration in the measurement-object gas; and
a reference-gas adjustment pump cell having a pump reference electrode disposed inside the element body so as to be in contact with the reference gas introduced to the reference-gas introduction portion, the reference-gas adjustment pump cell being configured to pump oxygen into a periphery of the pump reference electrode,
wherein the flow portion sensor cell has a voltage reference electrode disposed inside the element body so as to be in contact with the reference gas introduced to the reference-gas introduction portion.

5. The sensor element according to claim 1, further comprising:
an outer sensor cell having a voltage outer electrode disposed outside the element body, the outer sensor cell being configured to generate a voltage based on an oxygen concentration in the measurement-object gas outside the element body,
wherein the flow portion pump cell has a pump outer electrode disposed outside the element body.

6. The sensor element according to claim 2, further comprising:
an outer sensor cell having a voltage outer electrode disposed outside the element body, the outer sensor cell being configured to generate a voltage based on an oxygen concentration in the measurement-object gas outside the element body,
wherein the adjustment chamber pump cell has a pump outer electrode disposed outside the element body.

7. A gas sensor comprising:
the sensor element according to claim 1; and
a flow portion pump cell controller that causes the flow portion pump cell to pump out oxygen from the internal cavity or pump oxygen into the internal cavity by feedback-controlling the flow portion pump cell so that the voltage of the flow portion sensor cell reaches a target voltage.

8. A gas sensor comprising:
the sensor element according to claim 6;
an adjustment chamber pump cell controller that causes the adjustment chamber pump cell to pump out oxygen from the oxygen concentration adjustment chamber or pump oxygen into the oxygen concentration adjustment chamber by controlling the adjustment chamber pump cell so that an oxygen concentration in the oxygen concentration adjustment chamber reaches a predetermined low concentration; and
an oxygen concentration detector that detects an oxygen concentration in the measurement-object gas outside the element body based on the voltage of the outer sensor cell.

9. The sensor element according to claim 2, further comprising:
a reference-gas introduction portion disposed inside the element body, the reference-gas introduction portion being configured to introduce a reference gas serving as a reference for detecting a specific gas concentration in the measurement-object gas; and
a reference-gas adjustment pump cell having a pump reference electrode disposed inside the element body so as to be in contact with the reference gas introduced to the reference-gas introduction portion, the reference-gas adjustment pump cell being configured to pump oxygen into a periphery of the pump reference electrode,
wherein the flow portion sensor cell has a voltage reference electrode disposed inside the element body so as to be in contact with the reference gas introduced to the reference-gas introduction portion.

10. The sensor element according to claim 3, further comprising:
a reference-gas introduction portion disposed inside the element body, the reference-gas introduction portion being configured to introduce a reference gas serving as a reference for detecting a specific gas concentration in the measurement-object gas; and
a reference-gas adjustment pump cell having a pump reference electrode disposed inside the element body so as to be in contact with the reference gas introduced to the reference-gas introduction portion, the reference-gas adjustment pump cell being configured to pump oxygen into a periphery of the pump reference electrode,
wherein the flow portion sensor cell has a voltage reference electrode disposed inside the element body so as to be in contact with the reference gas introduced to the reference-gas introduction portion.

11. The sensor element according to claim 4, further comprising:
an outer sensor cell having a voltage outer electrode disposed outside the element body, the outer sensor cell being configured to generate a voltage based on an oxygen concentration in the measurement-object gas outside the element body,
wherein the flow portion pump cell has a pump outer electrode disposed outside the element body.

12. The sensor element according to claim 3, further comprising:
an outer sensor cell having a voltage outer electrode disposed outside the element body, the outer sensor cell being configured to generate a voltage based on an oxygen concentration in the measurement-object gas outside the element body,
wherein the adjustment chamber pump cell has a pump outer electrode disposed outside the element body.
